(19) United States
(12) Patent Application Publication
(10) Pub. No.: US 2025/0318721 A1
(43) Pub. Date: Oct. 16, 2025

(54) ENDOSCOPE CONTROL PROCESSING APPARATUS, ENDOSCOPE SYSTEM, METHOD OF OPERATING ENDOSCOPE CONTROL PROCESSING APPARATUS, AND ENDOSCOPE APPARATUS

(71) Applicant: OLYMPUS MEDICAL SYSTEMS CORP., Tokyo (JP)

(72) Inventor: Hidetaro KONO, Tokyo (JP)

(73) Assignee: OLYMPUS MEDICAL SYSTEMS CORP., Tokyo (JP)

(21) Appl. No.: 19/250,239

(22) Filed: Jun. 26, 2025

Related U.S. Application Data
(63) Continuation of application No. PCT/JP2022/048398, filed on Dec. 27, 2022.

(51) Int. Cl. A61B 1/045; A61B 1/00; A61B 1/05; G06T 7/80; G16H 40/63

(52) U.S. Cl. CPC A61B 1/045; A61B 1/00006; A61B 1/05; G06T 7/80; G16H 40/63; G06T 2207/10068

(57) ABSTRACT

Image signals are acquired in frame units from an endoscope including an image pickup device. The image pickup device has an electronic shutter function configured to control an exposure time. A processor performs light adjustment detection on an image signal to calculate a target value of an exposure time, calculates an electronic shutter control value from the target value, calculates an actual value of the exposure time from the electronic shutter control value, calculates an exposure time ratio between the actual value and the target value, acquires from the endoscope an image signal in a new frame picked up based on the electronic shutter control value, and adjusts a gain of the image signal in the new frame based on the exposure time ratio.

### CROSS REFERENCE TO RELATED APPLICATION

This application is a continuation application of PCT/JP2022/048398 filed on Dec. 27, 2022, the entire contents of which are incorporated herein by this reference.

The present disclosure relates to an endoscope control processing apparatus configured to acquire an image signal from an image pickup device having an electronic shutter function and process the image signal, an endoscope system, a method of operating the endoscope control processing apparatus, and an endoscope apparatus.

### BACKGROUND

An aperture, an electronic shutter, and a gain have been used for light adjustment control. The aperture performs the light adjustment control by changing the brightness of an optical image. The electronic shutter performs the light adjustment control by changing an exposure time, i.e., a charge accumulation time. The gain performs the light adjustment control by amplifying an image signal.

For example, Japanese Patent Application Laid-Open Publication No. 2018-14680 describes a technology for achieving a wide light adjustment dynamic range in a digital still camera. In the technology described in the publication, the following light adjustment control is performed while a state of an object changes from a dark state to a bright state.

When the object is at its darkest, the aperture is open, the gain is set to its maximum, and the degree to which the electronic shutter is slowed down relative to a frame rate is controlled. When the object is slightly dark, the aperture is open, the electronic shutter is set to the frame rate, and the gain is controlled between the maximum and 0. When the object is slightly bright, the electronic shutter is set to the frame rate, the gain is set to 0, and the aperture is controlled. When the object is bright, the gain is set to 0, the aperture is closed to its maximum, and the degree to which the electronic shutter makes faster than the frame rate is controlled.

In this way, in the above-described publication, the light adjustment control is performed in the order of “electronic shutter control”, “gain control”, “aperture control”, and “electronic shutter control”, as the state of the object changes from the dark state to the bright state. In other words, the technology changes which of the electronic shutter, the gain, or the aperture is used depending on the brightness of the object, when the light adjustment control is performed using only one of the electronic shutter, the gain, and the aperture. Therefore, the above-described publication does not describe control using two or more of the electronic shutter, the gain, and the aperture simultaneously.

### SUMMARY

An endoscope control processing apparatus according to one aspect of the present disclosure is an endoscope control processing apparatus configured to acquire image signals in frames from an endoscope including an image pickup device having an electronic shutter function configured to control an exposure time, and to process the image signals. The endoscope control processing apparatus includes a processor. The processor is configured to: perform light adjustment detection on an image signal acquired in a first frame to calculate an exposure time target value for a second frame that is later in time than the first frame; calculate an electronic shutter control value for the second frame based on the exposure time target value; calculate an actual exposure time from the electronic shutter control value; calculate an exposure time ratio between the actual exposure time and the exposure time target value; cause the image pickup device to pick up an image based on the electronic shutter control value, in order to acquire an image signal in the second frame from the endoscope; and adjust a gain based on the exposure time ratio in image processing on the image signal in the second frame.

An endoscope system according to one aspect of the present disclosure includes: an endoscope including an image pickup device having an electronic shutter function configured to control an exposure time; and an endoscope control processing apparatus including a processor configured to acquire image signals in frames from the endoscope, and to process the image signals. The processor is configured to: perform light adjustment detection on an image signal acquired in a first frame to calculate an exposure time target value for a second frame that is later in time than the first frame; calculate an electronic shutter control value for the second frame based on the exposure time target value; calculate an actual exposure time from the electronic shutter control value; calculate an exposure time ratio between the actual exposure time and the exposure time target value; cause the image pickup device to pick up an image based on the electronic shutter control value, in order to acquire an image signal in the second frame from the endoscope; and adjust a gain based on the exposure time ratio in image processing on the image signal in the second frame.

A method of operating an endoscope control processing apparatus according to one aspect of the present disclosure is a method of operating an endoscope control processing apparatus configured to acquire image signals in frames from an endoscope including an image pickup device having an electronic shutter function configured to control an exposure time, and to process the image signals. The method includes: performing light adjustment detection on an image signal acquired in a first frame to calculate an exposure time target value for a second frame that is later in time than the first frame; calculating an electronic shutter control value for the second frame based on the exposure time target value; calculating an actual exposure time from the electronic shutter control value; calculating an exposure time ratio between the actual exposure time and the exposure time target value; causing the image pickup device to pick up an image based on the electronic shutter control value, in order to acquire an image signal in the second frame from the endoscope; and adjusting a gain based on the exposure time ratio in image processing on the image signal in the second frame.

A non-transitory computer-readable recording medium according to one aspect of the present disclosure records a program configured to cause a computer configured to acquire image signals in frames from an endoscope including an image pickup device having an electronic shutter function configured to control an exposure time and to process the image signals, to perform endoscope control processing. The endoscope control processing includes: performing light adjustment detection on an image signal acquired in a first frame to calculate an exposure time target value for a second frame that is later in time than the first frame; calculating an electronic shutter control value for the second frame based on the exposure time target value; calculating an actual exposure time from the electronic shutter control value; calculating an exposure time ratio between the actual exposure time and the exposure time target value; causing the image pickup device to pick up an image based on the electronic shutter control value, in order to acquire an image signal in the second frame from the endoscope; and adjusting a gain based on the exposure time ratio in image processing on the image signal in the second frame.

A hardware processor of an endoscope apparatus according to one aspect of the present disclosure is configured to: generate a first image signal by performing image processing on a first image pickup signal, the first image pickup signal being generated by an image pickup device reading a charge corresponding to a first frame; perform light adjustment detection on the first image signal to determine a target exposure time in a second frame that is a frame later than the first frame; determine an electronic shutter control value for adjusting an exposure time in the second frame based on the target exposure time; calculate an exposure time ratio that is a ratio between an actual exposure time adjusted by the electronic shutter control value and the target exposure time in the second frame; generate a second image signal by performing image processing on a second image pickup signal, the second image pickup signal being generated by the image pickup device reading a charge corresponding to the second frame, the image pickup device having been controlled based on the electronic shutter control value in the second frame; and adjust a gain based on the calculated exposure time ratio in the second frame in the image processing on the second image pickup signal.

### BRIEF DESCRIPTION OF DRAWINGS

FIG. 1 is a view showing an appearance of an endoscope system in a first embodiment of the present disclosure;

FIG. 2 is a block diagram showing an electrical configuration of the endoscope system in the above first embodiment;

FIG. 3 is a block diagram showing a configuration example of an endoscope control processing apparatus in the above first embodiment;

FIG. 4 is a timing chart showing operation of the endoscope system during image pickup of a moving image in the above first embodiment;

FIG. 5 is a flowchart showing operation of the endoscope control processing apparatus during image pickup of a moving image in the above first embodiment;

FIG. 6 is a flowchart showing processing of electronic shutter control by the endoscope control processing apparatus in the above first embodiment;

FIG. 7 is a diagram showing an example of a table showing a correspondence between an automatic light adjustment brightness level and a light adjustment detection target value that are stored in a memory in the above first embodiment;

FIG. 8 is a graph showing a relationship between an exposure time and an electronic shutter control value in the above first embodiment;

FIG. 9 is a diagram showing the relationship between the exposure time and the electronic shutter control value in the above first embodiment;

FIG. 10 is a flowchart showing processing of flicker correction in Step S3 of FIG. 5 in the above first embodiment;

FIG. 11 is a flowchart showing processing of calculation of an exposure time ratio in Step S22 of FIG. 10 in the above first embodiment;

FIG. 12 is a graph showing a relationship between a brightness control value and the electronic shutter control value in the above first embodiment;

FIG. 13 is a graph showing the vicinity of a maximum value of the brightness control value in FIG. 12 enlarged, along with the exposure time in the above first embodiment;

FIG. 14 is a graph showing the vicinity of a minimum value of the brightness control value in FIG. 12 enlarged, along with the exposure time in the above first embodiment;

FIG. 15 is a graph showing a ratio of a change amount of the exposure time when the electronic shutter control value changes by one line, to the exposure time in the above first embodiment;

FIG. 16 is a graph showing the vicinity of the minimum value of the brightness control value enlarged and showing how the exposure time that changes discretely is substantially corrected by a gain in the above first embodiment;

FIG. 17 is a block diagram showing an electrical configuration of an endoscope system in a second embodiment of the present disclosure;

FIG. 18 is a flowchart showing processing of flicker correction in Step S3 of FIG. 5 in the above second embodiment;

FIG. 19 is a view showing a configuration example of an endoscope control processing apparatus serving also as a light source device in an additional technology 1;

FIG. 20 is a block diagram showing a configuration example of a control circuit, a light source drive circuit, and a light source in the additional technology 1;

FIG. 21 is a diagram showing an example of a light emission mode of the endoscope control processing apparatus serving also as the light source device in the additional technology 1;

FIG. 22 is a view showing a more specific configuration example of the light source drive circuit and the light source in the additional technology 1;

FIG. 23 is a graph showing an example of ranges in which driving current detected by a current detection circuit in each light emission mode is normal and abnormal in the additional technology 1;

FIG. 24 is a timing chart showing an example of timing at which the current detection circuit performs current detection in the additional technology 1;

FIG. 25 is a view showing an example in which one current detection circuit is applied in a configuration in which a plurality of light-emitting elements are driven by a plurality of light-emitting element drivers respectively in the additional technology 1;

FIG. 26 is a view showing an example in which one current detection circuit is applied in a configuration in which the plurality of light-emitting elements are driven by one light-emitting element driver in the additional technology 1;

FIG. 27 is a view showing an example in which one current detection circuit is applied in a configuration in which one light-emitting element is driven by the plurality of light-emitting element drivers in the additional technology 1;

FIG. 28 is a view showing a configuration example of an endoscope control processing apparatus serving also as a light source device in an additional technology 2;

FIG. 29 is a graph showing a first example of conversion parameters for converting a current control value I into pulse brightness L in the additional technology 2;

FIG. 30 is a graph showing a second example of conversion parameters for converting the current control value I into the pulse brightness L in the additional technology 2;

FIG. 31 is a timing chart for explaining exposure unevenness between frames when an image pickup device is a global shutter type in the additional technology 2;

FIG. 32 is a timing chart for explaining the exposure unevenness between lines when the image pickup device is a rolling shutter type in the additional technology 2;

FIG. 33 is a view showing a configuration example of an endoscope control processing apparatus serving also as a light source device in an additional technology 3;

FIG. 34 is a graph showing an example of a function formula for calculating a color correction value from a light adjustment control value in the additional technology 3.

FIG. 35 is a view showing a configuration example of an endoscope processor configured to supply power source for a sound input circuit using an isolator element in an additional technology 4;

FIG. 36 is a graph for explaining that an amplification line with a large gain is used when an input sound pressure to a microphone is a threshold value or less, and an amplification line with a small gain is used when the input sound pressure is greater than the threshold value in the additional technology 4; and

FIG. 37 is a view showing a configuration example of the endoscope processor including a plurality of amplification mechanisms with different amplification degrees in the additional technology 4.

### DETAILED DESCRIPTION OF EMBODIMENTS

Embodiments of the present application will be described below with reference to the drawings. However, the present application is not necessarily limited to the embodiments described below.

Note that in the description of the drawings, like or corresponding elements are denoted with the same reference signs as appropriate. It should be noted that the drawings are schematic, and the relationship between lengths of respective elements, a ratio of the lengths of respective elements, and the numbers of respective elements in one drawing may be different from the actual ones in order to simplify the description. Furthermore, there may be parts where the relationship and the ratio of the lengths differ between the plurality of drawings.

### First Embodiment

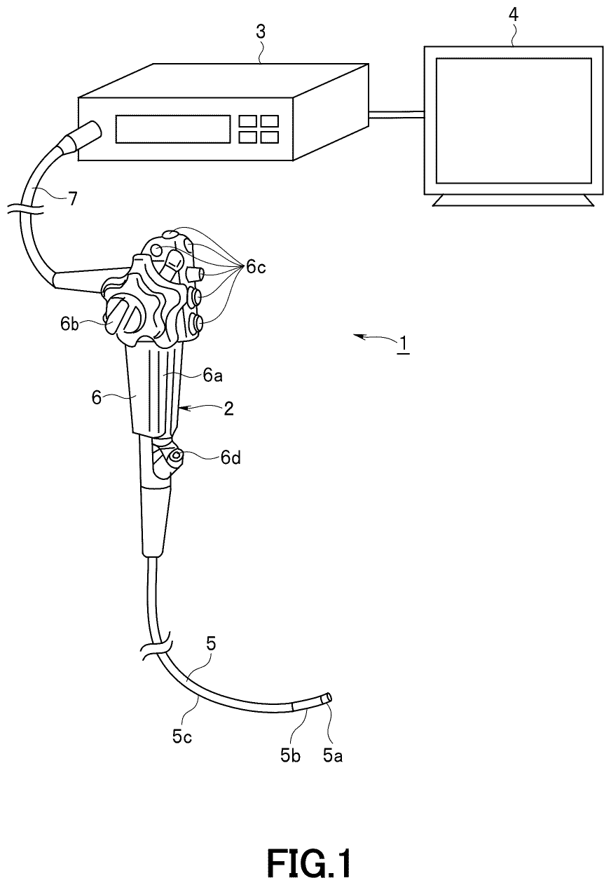

FIG. 1 to FIG. 16 show a first embodiment of the present disclosure. FIG. 1 is a view showing an appearance of an endoscope system 1.

The endoscope system 1 includes an endoscope 2, an endoscope control processing apparatus 3, and a monitor 4.

The endoscope 2 includes an insertion portion 5, an operation portion 6, and a universal cable 7.

The insertion portion 5 is a long slender part configured to be inserted into a body of a subject. Note that the subject into which the insertion portion 5 is inserted is assumed to be the human body as an example, but it is not limited to the human body and may be a living organism such as an animal, or even an inanimate object such as a machine or a building.

The insertion portion 5 includes a distal-end constituting portion 5a, a bending portion 5b, and a flexible tube portion 5c in this order from the distal end side toward the proximal end side.

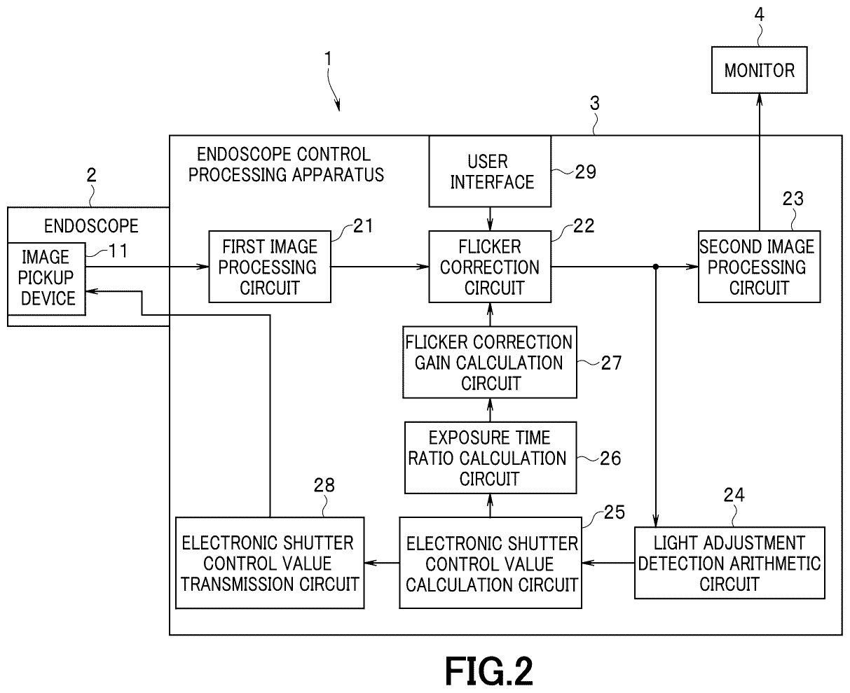

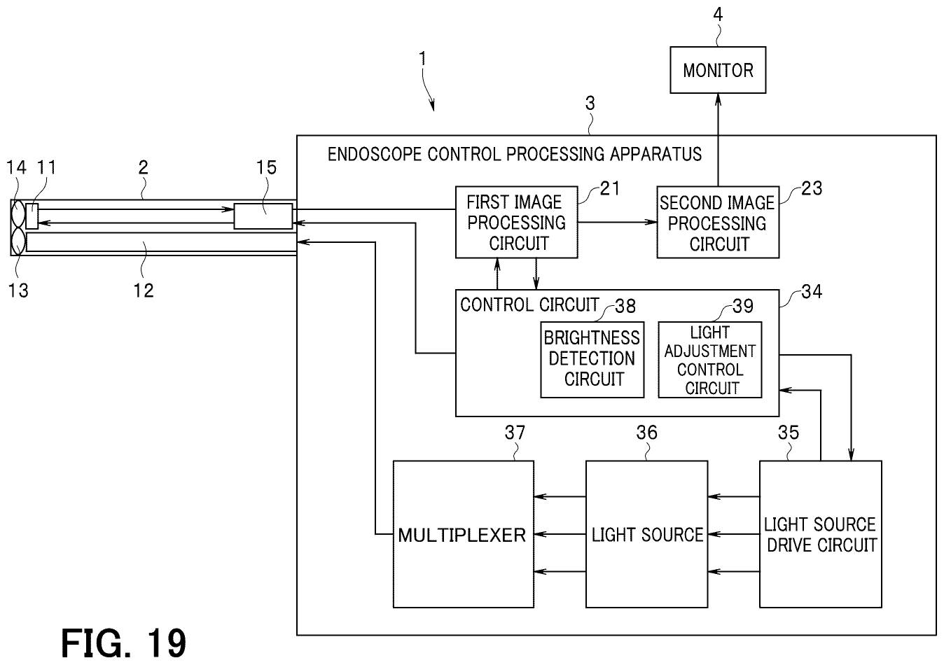

The endoscope 2 is configured as an electronic endoscope, and is provided with an image pickup system in the distal-end constituting portion 5a. The image pickup system includes an objective lens 14 (see FIG. 19) that forms an optical image of the subject, and an image pickup device 11 (see FIG. 2, etc.) that photoelectrically converts the optical image formed by the objective lens 14 and outputs an electric signal. The image pickup device 11 generates image signals in frame units and outputs the image signals to the endoscope control processing apparatus 3.

Note that the image pickup device 11 is not limited to being provided in the distal-end constituting portion 5a of the insertion portion 5. For example, a configuration in which a relay optical system is provided in the insertion portion 5 and the operation portion 6 and a camera head is attached to the operation portion 6 may be adopted. In this configuration, the optical image formed by the objective lens 14 is transmitted by the relay optical system, and picked up by the image pickup device 11 in the camera head.

The bending portion 5b is, for example, a part bendable in two directions, such as up and down, or four directions, such as up, down, left, and right. The bending portion 5b is disposed on the proximal end side of the distal-end constituting portion 5a. When the bending portion 5b is bent, the direction of the distal-end constituting portion 5a changes, and an irradiation direction of illumination light and an observation direction of the image pickup system change. In addition, the bending portion 5b is bent in order to improve insertability of the insertion portion 5 in the subject.

The flexible tube portion 5c is a tube portion having flexibility. The flexible tube portion 5c is disposed on the proximal end side of the bending portion 5b. Note that, in the present embodiment, an example in which the endoscope 2 is a flexible endoscope having the flexible tube portion 5c is given. However, the endoscope 2 may be a rigid endoscope having a configuration in which a part corresponding to the flexible tube portion 5c is rigid. In addition, the endoscope 2 may be any type of the following: a type in which the entirety of the endoscope 2 is disposable; a type in which the entirety is reused after reprocessing; or a type in which a part of the endoscope 2 is disposable.

The operation portion 6 is disposed on the proximal end side of the insertion portion 5. The operation portion 6 includes a grasping portion 6a, a bending operation knob 6b, operation buttons 6c, and a treatment instrument insertion port 6d.

The grasping portion 6a is a part for a user to grasp the endoscope 2 with the palm.

The bending operation knob 6b is an operation device for operating bending of the bending portion 5b. The bending operation knob 6b is operated by using, for example, the thumb of the hand grasping the grasping portion 6a. The bending operation knob 6b is connected to the bending portion 5b with bending wires. When the bending operation knob 6b is operated, the bending wires are pulled, thereby causing the bending portion 5b to bend.

The operation buttons 6c include a plurality of buttons for operating the endoscope 2. Some examples of the operation buttons 6c include a gas/liquid feeding button, a suction button, and a button related to image pickup.

The treatment instrument insertion port 6d is a proximal end side opening of a treatment instrument channel disposed inside the insertion portion 5 to the operation portion 6. When a treatment instrument is inserted from the treatment instrument insertion port 6d, the distal end of the treatment instrument protrudes from a distal end side opening of the treatment instrument channel, the distal end side opening being provided at the distal-end constituting portion 5a. In this state, various kinds of treatment are performed on the subject with the treatment instrument.

The universal cable 7 is extended, for example, from a side surface on the proximal end side of the operation portion 6, and connected to the endoscope control processing apparatus 3.

The endoscope control processing apparatus 3 receives an image signal in the frame unit from the image pickup device 11. The endoscope control processing apparatus 3 performs image processing on the acquired image signal and outputs the processed image signal to the monitor 4. In addition, the endoscope control processing apparatus 3 controls the endoscope 2. In other words, the endoscope control processing apparatus 3 serves also as an endoscopic image processing apparatus configured to process the image signal acquired by the endoscope 2, and an endoscope control apparatus configured to control the endoscope 2.

The monitor 4 is a display device configured to receive the image signal and display an endoscopic image. Note that the monitor 4 is not required to have a configuration unique to the endoscope system 1, and the monitor 4 provided separately from the endoscope system 1 may be used by connecting the monitor 4 to the endoscope control processing apparatus 3.

FIG. 2 is a block diagram showing an electrical configuration of the endoscope system 1.

The endoscope 2 includes the image pickup device 11 as described above. The image pickup device 11 is a two-dimensional image pickup device in which a plurality of pixels are arranged in a two-dimensional array in a matrix direction. An arrangement in a row direction of the pixels is referred to as a line. Examples of the image pickup device 11 include a charge coupled device (CCD) imager and a complementary metal oxide semiconductor (CMOS) imager.

The image pickup device 11 photoelectrically converts the optical image of the subject to generate and output the image signal. The image pickup device 11 has an electronic shutter function that electronically controls an exposure time. The image pickup device 11 realizes a state in which no charge is accumulated, that is, a light-shielded state, by discharging (sweeping out) the charge of the pixels. When the electronic shutter stops discharging the charge of the pixels, an accumulation of the charge is started. The time from the start of the accumulation of the charge until the accumulated charge is read out is the exposure time. In the electronic shutter function, the discharge of the charge of the pixels to realize the light-shielded state is performed, for example, on line-by-line basis.

An exposure period in one frame is the longest period that can be used for exposure within a frame period. Thus, in a case where the exposure period and a readout period are separated, and the image pickup device 11 cannot perform the exposure in the readout period, the exposure period is a period excluding the readout period, etc., from the frame period.

A time obtained by dividing the exposure period in one frame by the total number of lines provided in the image pickup device 11 is referred to as a specific time. The electronic shutter function of the image pickup device 11 is capable of controlling the exposure time in units of the specific time and in integer multiples of the specific time. Therefore, the exposure time is controlled discretely by the electronic shutter function.

Specifically, it is supposed that the exposure period in one frame is Tep, the total number of lines provided in the image pickup device 11 is N. Then, the specific time which is the unit by which the image pickup device 11 can control the exposure time, is (Tep/N). Note that the exposure period Tep is determined depending on the specification of the image pickup device 11.

The endoscope control processing apparatus 3 includes a first image processing circuit 21, a flicker correction circuit 22, a second image processing circuit 23, a light adjustment detection arithmetic circuit 24, an electronic shutter control value calculation circuit 25, an exposure time ratio calculation circuit 26, a flicker correction gain calculation circuit 27, an electronic shutter control value transmission circuit 28, and a user interface 29.

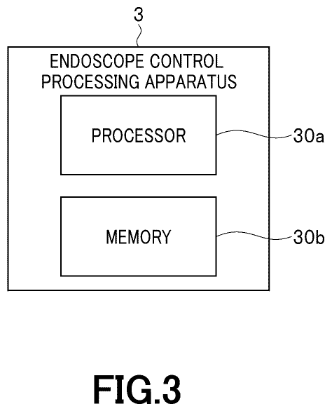

FIG. 3 is a block diagram showing a hardware configuration example of the endoscope control processing apparatus 3. The endoscope control processing apparatus 3 in FIG. 3 includes a processor 30a and a memory 30b. The processor 30a and the memory 30b are hardware.

Each circuit of the endoscope control processing apparatus 3 shown in FIG. 2 may be configured by electronic circuits, which are hardware. In addition, the entirety or a part of each circuit of the endoscope control processing apparatus 3 shown in FIG. 2 may be configured by the processor 30a and the memory 30b as shown in FIG. 3. The processor 30a is configured by an application specific integrated circuit (ASIC) including a central processing unit (CPU), etc., a field programmable gate array (FPGA), or the like. The memory 30b stores a processing program that causes the processor 30a to realize functions of each circuit. The processor 30a reads and executes the processing program stored in the memory 30b, to realize the functions of each circuit in the endoscope control processing apparatus 3.

The first image processing circuit 21 receives the image signal outputted by the image pickup device 11 and performs first image processing. The first image processing includes, for example, amplification processing, demosaic processing, noise removal processing, etc.

The flicker correction circuit 22 amplifies the image signal outputted from the first image processing circuit 21 with a flicker correction gain received from the flicker correction gain calculation circuit 27, and performs image processing of correcting flickers of a frame image.

The second image processing circuit 23 performs second image processing on the image signal outputted from the flicker correction circuit 22. The second image processing includes, for example, shading correction processing, white balance processing, contrast correction processing, gamma conversion processing, format conversion processing, etc. In addition, the second image processing circuit 23 may superimpose various information, such as character information and guide information, on the image signal. The second image processing circuit 23 outputs to monitor 4 the image signal converted into a format suitable for the monitor 4.

Note that although several examples of the first image processing and several examples of the second image processing are described above, processing included in the first image processing and the second image processing is not limited to those. Furthermore, which processing is included in the first image processing performed in the first stage in the flicker correction circuit 22 and which other processing is included in the second image processing performed in the latter stage in the flicker correction circuit 22 is not limited to the example described above.

The light adjustment detection arithmetic circuit 24 performs light adjustment detection on the image signal outputted from the first image processing circuit 21 and acquires a light adjustment detection value.

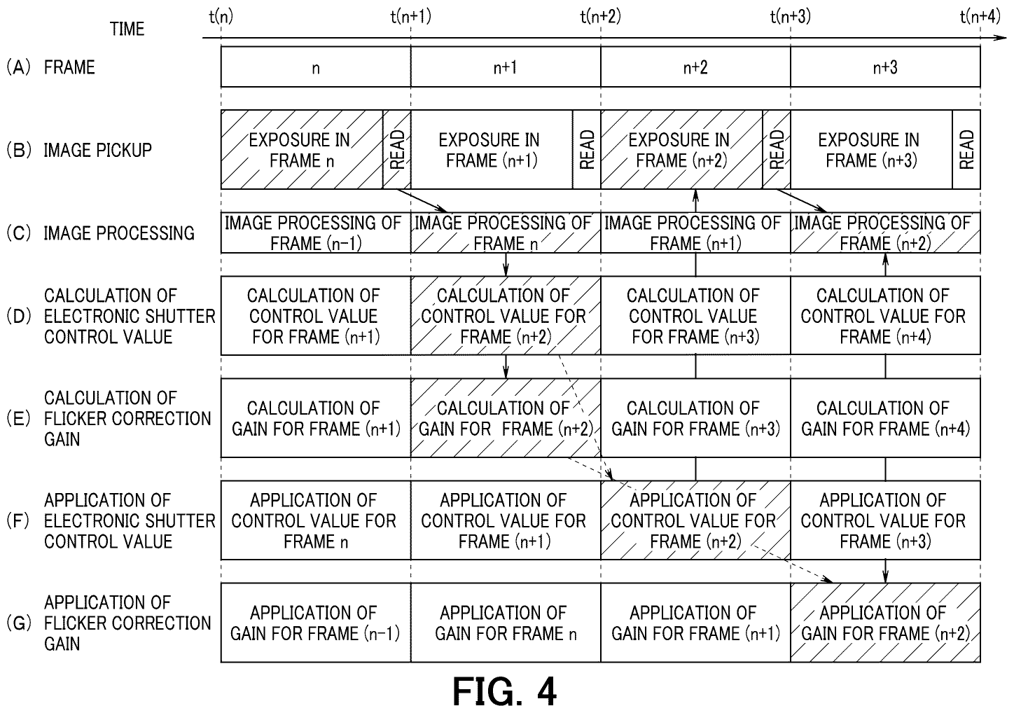

The light adjustment detection arithmetic circuit 24 calculates an exposure time target value of a second frame, which is later in time than a first frame, based on the light adjustment detection value of the image signal acquired in the first frame. Note that in FIG. 4, which will be described later, the first frame is described as a frame n and the second frame is described as a frame (n+2), specifically. The exposure time target value of the second frame is calculated so that the light adjustment detection value of the image signal acquired in the second frame is a light adjustment detection target value. Here, an automatic light adjustment brightness level that provides the light adjustment detection target value can be set by a user through the user interface 29, as described later.

The electronic shutter control value calculation circuit 25 receives the exposure time target value from the light adjustment detection arithmetic circuit 24 and calculates an electronic shutter control value applied to the second frame. The exposure time target value is calculated as an arbitrary value that is not discrete. In contrast, the electronic shutter control value is calculated as a value representing a discrete exposure time (an integer multiple of a specific time as described above, for example).

The exposure time ratio calculation circuit 26 acquires the exposure time target value and the electronic shutter control value from the electronic shutter control value calculation circuit 25. The exposure time ratio calculation circuit 26 calculates a ratio of the electronic shutter control value to the exposure time target value as an exposure time ratio. Note that in FIG. 2, the exposure time ratio calculation circuit 26 acquires the exposure time target value from the electronic shutter control value calculation circuit 25, but the present disclosure is not limited thereto. The exposure time ratio calculation circuit 26 may receive the exposure time target value from the light adjustment detection arithmetic circuit 24.

The flicker correction gain calculation circuit 27 acquires the exposure time ratio from the exposure time ratio calculation circuit 26, calculates an inverse of the exposure time ratio as a flicker correction gain, and transmits the flicker correction gain to the flicker correction circuit 22.

Note that in the above description, the exposure time ratio is calculated as the ratio of the electronic shutter control value to the exposure time target value, but it is not limited thereto, and the exposure time ratio may be calculated as a ratio of the exposure time target value to the electronic shutter control value. In this case, the flicker correction gain calculation circuit 27 may transmit the exposure time ratio as the flicker correction gain to the flicker correction circuit 22. In this case, the exposure time ratio calculation circuit 26 can serve also as the flicker correction gain calculation circuit 27, thereby simplifying the configuration.

The electronic shutter control value transmission circuit 28 receives the electronic shutter control value from the electronic shutter control value calculation circuit 25, and transmits the electronic shutter control value to the image pickup device 11. In addition, the endoscope control processing apparatus 3 transmits an operation clock and power to the image pickup device 11. The image pickup device 11 performs image pickup so that the subject is exposed according to the electronic shutter control value, and generates the image signal. The image pickup of a moving image by the image pickup device 11 is performed sequentially in frame units (frames) as described above. With this, the image signals in frame units related to the moving image are outputted sequentially from the endoscope 2 to the endoscope control processing apparatus 3.

The user interface 29 is an interface operated by a user to make various setting for the endoscope system 1. For example, the user interface 29 is used to set the exposure time control by the electronic shutter to on or off. In the present embodiment, the description is made on the assumption that the exposure time control by the electronic shutter is set to on. The user can set the automatic light adjustment brightness level through the user interface 29.

The monitor 4 receives the image signal outputted from the second image processing circuit 23 and displays an endoscopic image.

FIG. 4 is a timing chart showing operation of the endoscope system 1 during image pickup of a moving image. In FIG. 4, the frame number is indicated in (A), the operation of the image pickup is indicated in (B), the operation of the image processing is indicated in (C), the operation of the electronic shutter control value calculation is indicated in (D), the operation of the flicker correction gain value calculation is indicated in (E), the operation of the electronic shutter control value application is indicated in (F), and the operation of the flicker correction gain application is indicated in (G).

As shown in (A) of FIG. 4, the time t(n) to t(n+1) is the period of the frame n, the time t(n+1) to t(n+2) is the period of the frame (n+1), the time t(n+2) to t(n+3) is the period of the frame (n+2), and the time t(n+3) to t(n+4) is the period of the frame (n+3).

As shown in (B) of FIG. 4, in each frame, the exposure is performed in the first stage of the frame period, and the image signal is read out in the latter stage of the frame period.

During the period of the frame n, the image pickup device 11 performs exposure of the image in the frame n and reads out the image signal. The image signal in the frame n read out by the image pickup device 11 is an example of a first image pickup signal. Hereinafter, a series of processing performed by the endoscope control processing apparatus 3 based on the image signal acquired during the period of the frame n will be described. In FIG. 4, the corresponding blocks are hatched and each block is connected by an arrow.

During the period of the frame (n+1), the first image processing circuit 21 and the second image processing circuit 23 perform the image processing (C) on the image signal in the frame n. In addition, the light adjustment detection arithmetic circuit 24 and the electronic shutter control value calculation circuit 25 perform the electronic shutter control value calculation (D) for image pickup in the frame (n+2). Furthermore, the exposure time ratio calculation circuit 26 and the flicker correction gain calculation circuit 27 perform the flicker correction gain calculation (E) on the image acquired in the frame (n+2).

During the period of the frame (n+2), the electronic shutter control value transmission circuit 28 transmits the electronic shutter control value to the image pickup device 11 (F). The transmitted value is the electronic shutter control value for the frame (n+2) to be applied to the image pickup device 11. The image pickup device 11 performs exposure of the image in the frame (n+2) with the received electronic shutter control value, and reads out the generated image signal (B). The image signal in the frame (n+2) read out by the image pickup device 11 is an example of a second image pickup signal.

During the period of the frame (n+3), the first image processing circuit 21 processes the image signal acquired in the period of the frame (n+2) (C). The flicker correction circuit 22 applies the flicker correction gain for the frame (n+2) to the processed image signal to correct flickers in the image (G). Furthermore, the second image processing circuit 23 processes the flicker-corrected image signal (C).

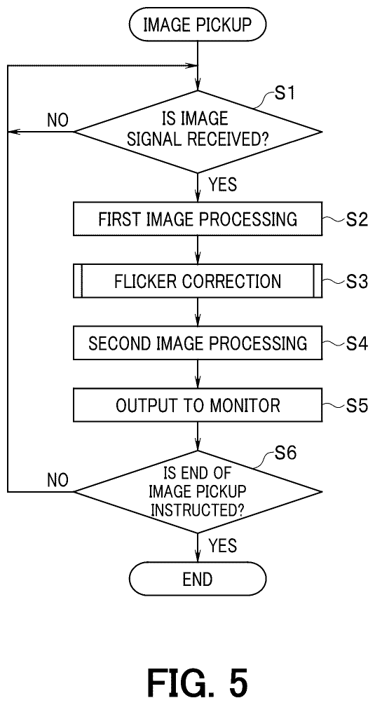

FIG. 5 is a flowchart showing operation of the endoscope control processing apparatus 3 during the image pickup of the moving image.

When the image pickup of the moving image starts, the endoscope control processing apparatus 3 waits to receive an image signal in frame units from the image pickup device 11 of the endoscope 2 (Step S1).

When the image signal is received, the first image processing circuit 21 performs the first image processing on the image signal (Step S2).

The flicker correction circuit 22 performs the flicker correction on the image signal outputted from the first image processing circuit 21 (Step S3).

The second image processing circuit 23 performs the second image processing on the image signal outputted from the flicker correction circuit 22 (Step S4).

The second image processing circuit 23 outputs the processed image signal to the monitor 4 (Step S5).

The endoscope control processing apparatus 3 determines whether the user instructs the end of the image pickup of the moving image (Step S6), and if the end is not instructed, the endoscope control processing apparatus 3 returns to Step S1 and waits to receive the image signal in the next frame. If the end is instructed, the image pickup processing is ended.

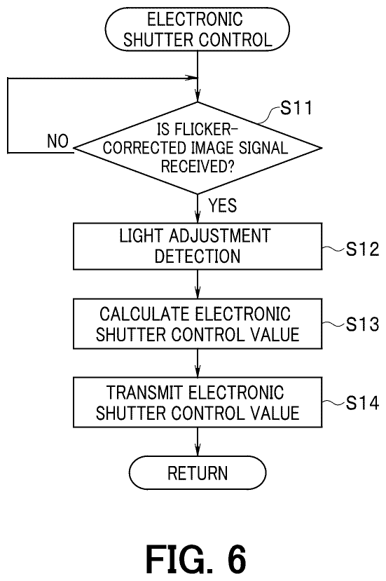

FIG. 6 is a flowchart showing the processing of the electronic shutter control by the endoscope control processing apparatus 3. The processing in FIG. 6 is performed in parallel with, for example, the processing in FIG. 5, for performing the image processing for displaying on the monitor 4.

When the processing in FIG. 6 is performed from main processing not shown, the light adjustment detection arithmetic circuit 24 waits to receive the flicker-corrected image signal from the flicker correction circuit 22 (Step S11).

When the image signal is received, the light adjustment detection arithmetic circuit 24 performs the light adjustment detection based on the image signal, and calculates the exposure time target value (Step S12).

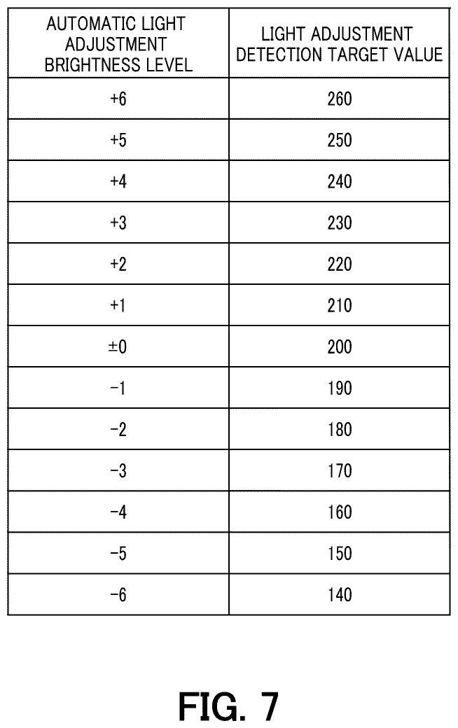

FIG. 7 is a diagram showing an example of a table showing a correspondence between the automatic light adjustment brightness level and the light adjustment detection target value that is stored in the memory 30b.

The automatic light adjustment brightness level set by the user through the user interface 29 is set based on how much brighter (level=+1, +2, +3, . . . ) or darker (level=−1, −2, −3, . . . ), etc., the image brightness is to be, compared to the standard brightness (level=0). The correspondence between the automatic light adjustment brightness level and the light adjustment detection target value is stored in advance in the memory 30b in the form of a table, a graph, a function, or the like, for example.

Here, the correspondence between the automatic light adjustment brightness level and the light adjustment detection target value differs depending on whether the endoscope 2 connected to the endoscope control processing apparatus 3 is a flexible endoscope, a type that uses a camera head, or the like. For this reason, the correspondence for each model of the endoscope 2 is stored in the memory 30b. FIG. 7 shows an example of the correspondence when the endoscope 2 is a flexible endoscope.

The light adjustment detection arithmetic circuit 24 refers to the memory 30, and determines the light adjustment detection target value based on the automatic light adjustment brightness level set through the user interface 29. Note that when the automatic light adjustment brightness level is not set through the user interface 29, the light adjustment detection target value is automatically set to a value corresponding to the standard brightness (level=0).

Furthermore, the light adjustment detection arithmetic circuit 24 extracts, for example, a luminance signal from the image signal received from the flicker correction circuit 22, and calculates as the light adjustment detection value an average value (or weighted average value, peak value of the luminance histogram, median value, or the like) of luminance signal values within a light adjustment region set for the entire image or a part of the image.

The light adjustment detection arithmetic circuit 24 calculates, for example, a first detection ratio by dividing the light adjustment detection value by the light adjustment detection target value, and calculates the exposure time target value by dividing the exposure time target value in the first frame (the frame n in FIG. 4, for example) by the first detection ratio. The exposure time target value is expressed in units of seconds (sec), for example.

As a numerical example, suppose that the exposure time target value in the first frame is 0.001 (sec) and the first detection ratio is 0.5. Then, the exposure time target value is calculated as 0.001/0.5=0.002 (sec).

Note that the light adjustment detection arithmetic circuit 24 may calculate a second detection ratio by dividing the light adjustment detection target value by the light adjustment detection value, instead of using the first detection ratio. In this case, the light adjustment detection arithmetic circuit 24 may calculate the exposure time target value by multiplying the exposure time target value in the first frame by the second detection ratio.

The electronic shutter control value calculation circuit 25 receives the exposure time target value from the light adjustment detection arithmetic circuit 24, and calculates the electronic shutter control value (Step S13). The relationship between the electronic shutter control value and the exposure time is predetermined depending on the specification of the image pickup device 11, as described below.

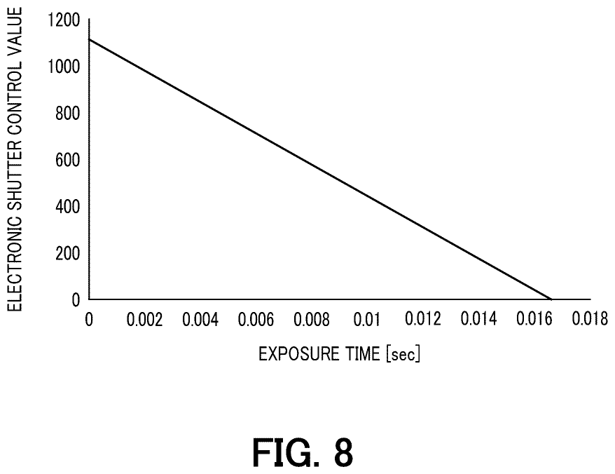

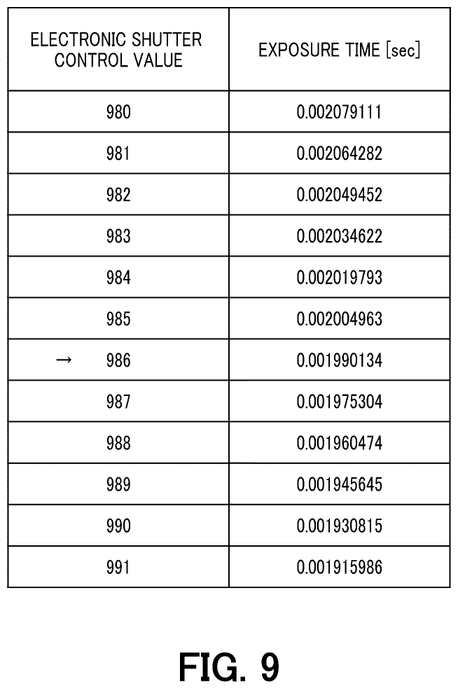

FIG. 8 is a graph showing the relationship between the exposure time and the electronic shutter control value. FIG. 9 is a diagram showing the relationship between the exposure time and the electronic shutter control value.

Note that in the present embodiment including FIG. 8 and FIG. 9, description will be made using a numerical example in which the total number of lines N of the image pickup device 11 is 1120 and the frame rate is 60 (fps). However, needless to say, it is not limited to this numerical example.

The exposure time is controlled by an integer multiple of a specific time (Tep/N) as described above. Therefore, when n is a positive integer less than or equal to N, the exposure time is expressed by n×(Tep/N).

It is supposed that the maximum value of the exposure time is Tmax. The maximum value Tmax of the exposure time is a value when n=N, and Tmax=N×(Tep/N)=Tep. In other words, when all lines are in the exposed state and the exposure time matches the exposure period Tep, the exposure time becomes the maximum value Tmax.

It is supposed that the minimum value of the exposure time is Tmin. The minimum value Tmin of the exposure time is not necessarily the time (Tep/N) corresponding to n=1. Depending on the specification of the image pickup device 11, n is set to an integer value of 1 or more, such as n=4, for example. When the minimum value of n that can be taken depending on the specification of the image pickup device 11 is n(min), the minimum value Tmin of the exposure time is Tmin=n(min)×(Tep/N). The minimum value Tmin of the exposure time corresponds to the electronic shutter being fully closed (the state in which the number of the exposure lines is minimum: the state in which the number of light-shielded lines is maximum).

Therefore, the exposure time n×(Tep/N) is controlled within a range of n=n(min), {n(min)+1}, . . . , (N−1), N.

The endoscope control processing apparatus 3 uses ES=(N−n) as the electronic shutter control value ES for the image pickup device 11. The electronic shutter control value ES is a value representing how many lines of the total number of lines N that are in the light-shielded state. Therefore, as shown in FIG. 8 and FIG. 9, as the exposure time becomes shorter, the electronic shutter control value (the number of lines to be in the light-shielded state) increases. However, it is not limited this, and the electronic shutter control value may be represented by the number of lines to be in the exposed state.

It is supposed that the exposure time target value received by the electronic shutter control value calculation circuit 25 from the light adjustment detection arithmetic circuit 24 is Tgt. In addition, the minimum value of the electronic shutter control value ES is ESmin, and the maximum value of the electronic shutter control value ES is ESmax.

The minimum value ESmin of the electronic shutter control value ES corresponds to the maximum value Tmax of the exposure time described above, and ESmin=0.

The maximum value ESmax of the electronic shutter control value ES corresponds to the minimum value Tmin of the exposure time described above, and ESmax={N−n (min)}.

As described above, the exposure time of each line in the image pickup device 11 is controlled by an integer multiple of the specific time (Tep/N). Therefore, the electronic shutter control value calculation circuit 25 calculates the electronic shutter control value ES capable of realizing “the exposure time closest to the exposure time target value Tgt” as follows.

Specifically, the electronic shutter control value calculation circuit 25 calculates the electronic shutter control value ES from the exposure time target value Tgt using a ceiling function ceil(x) according to the following Equation 1. Here, the ceiling function ceil(x) is a function that returns the smallest integer that is greater than or equal to x for the real number x (that is, a function that rounds up the decimal part), as is well known.

$$ES = ceil ( ESmax - α \times \{ Tgt - Tmin \} ) \qquad ( Equation 1 )$$

Here, −α represents an inclination of the graph shown in FIG. 8, and α is given by the following Equation 2.

$$α = ( ESmax - ESmin ) / Tmax - Tmin ) \qquad ( Equation 2 )$$

Note that here, the ceiling function ceil(x) is used to round up the decimal point, but instead the floor function floor(x) may be used to round down the decimal point.

However, as described above, Tmax=Tep and ESmin=0. Therefore, Equation 2 can be expressed as the following Equation 2′.

$$α = ESmax / ( Tep - Tmin ) \qquad ( Equation {2}^{′} )$$

Note that when n (min)=4 as described in the above example, the maximum value ESmax of the electronic shutter control value ES is (N−4), and the minimum value Tmin of the exposure time is 4×(Tep/N).

Thus, using Tmin, ESmax, and Tep determined depending on the specification of the image pickup device 11, and a determined from these, the electronic shutter control value ES is calculated from the exposure time target value Tgt using Equation 1.

Note that the number of lines n in the exposed state is obtained by subtracting the electronic shutter control value ES representing the number of lines to be in the light-shielded state from the total number of lines N, and n=(N−ES).

As in the numerical example described above, when the exposure time target value Tgt calculated by the light adjustment detection arithmetic circuit 24 is 0.002 (sec), the electronic shutter control value ES capable of realizing the exposure time not exceeding 0.002 (sec) but closest to 0.002 (sec) is calculated as 986 (lines) (see the section with the arrow in FIG. 9).

Therefore, the electronic shutter control value calculation circuit 25 may calculate the electronic shutter control value ES using the table shown in FIG. 9, instead of Equation 1. Equations, and the like that give the table shown in FIG. 9 or the graph shown in FIG. 8 may be stored in the memory 30b in advance, and be read out by the electronic shutter control value calculation circuit 25.

The electronic shutter control value transmission circuit 28 receives the electronic shutter control value ES from the electronic shutter control value calculation circuit 25, and transmits the electronic shutter control value ES to the image pickup device 11 (Step S14). As a result, the image pickup device 11 performs the electronic shutter control based on the electronic shutter control value ES in the next frame (the frame (n+2) in FIG. 4, for example).

After performing the processing in Step S14, the processing return to the main processing not shown.

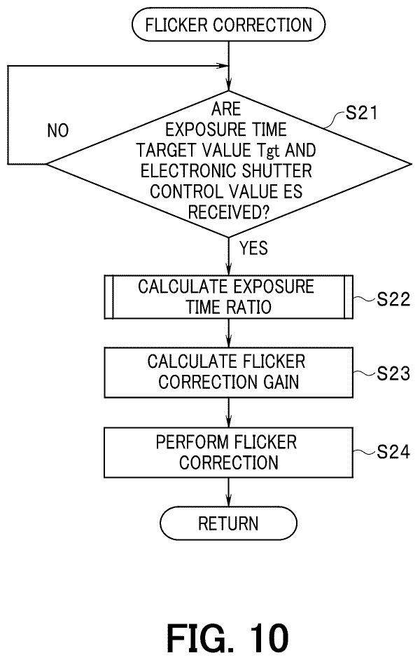

FIG. 10 is a flowchart showing the processing of the flicker correction in Step S3 of FIG. 5.

When the endoscope control processing apparatus 3 enters the processing of the flicker correction in Step S3 of FIG. 5, the processing shown in FIG. 10 is performed. Then, the exposure time ratio calculation circuit 26 waits to receive the exposure time target value Tgt and the electronic shutter control value ES from the electronic shutter control value calculation circuit 25 (Step S21).

When receiving the exposure time target value Tgt and the electronic shutter control value ES, the exposure time ratio calculation circuit 26 calculates the exposure time ratio er (Step S22).

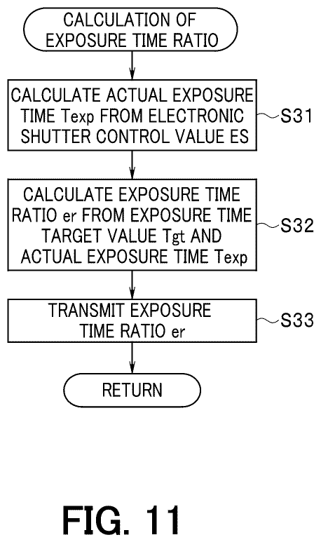

FIG. 11 is a flowchart showing the processing of the exposure time ratio calculation in Step S22 of FIG. 10.

The exposure time ratio calculation circuit 26 calculates an actual exposure time Texp using the electronic shutter control value ES and the above-described a according to the following Equation 3 (Step S31).

$$Texp = - ES / α + Tmax \qquad ( Equation 3 )$$

As described above, since Tmax=Tep, Equation 3 can be expressed as the following Equation 3′.

$$Texp = - ES / α + Tep \qquad ( Equation {3}^{′} )$$

Note that the exposure time ratio calculation circuit 26 is not limited to calculating the actual exposure time Texp using the equation, but may calculate the actual exposure time Texp by referring to the diagram of FIG. 9 (or the graph of FIG. 8).

As in the numerical example described above, when the electronic shutter control value ES is 986 (lines), the actual exposure time Texp is 0.001990134 (sec), as can be seen from the section with the arrow in FIG. 9, for example.

The exposure time ratio calculation circuit 26 calculates the exposure time ratio er using the exposure time target value Tgt and the actual exposure time Texp according to the following Equation 4 (Step S32).

$$er = Texp / Tgt \qquad ( Equation 4 )$$

As in the numerical example described above, when the exposure time target value Tgt is 0.002 (sec) and the actual exposure time Texp is 0.001990134 (sec), the exposure time ratio is as follows.

$$er = 0.001990134 / 0.002 = 0.995067$$

Therefore, the actual exposure time Texp is 0.4933% shorter than the exposure time target value Tgt.

The exposure time ratio calculation circuit 26 transmits the calculated exposure time ratio er to the flicker correction gain calculation circuit 27 (Step S33). After performing Step S33, the processing is returned to FIG. 10.

In the processing of FIG. 10, the flicker correction gain calculation circuit 27 calculates the inverse of the exposure time ratio er received from the exposure time ratio calculation circuit 26 as a flicker correction gain Ga as shown in the following Equation 5 (Step S23).

$$Ga = 1 / er \qquad ( Equation 5 )$$

As in the numerical example described above, when the exposure time ratio er is 0.995067, the flicker correction gain Ga is as follows.

$$Ga = 1 / 0.995067 = 1.004957$$

The flicker correction gain calculation circuit 27 transmits the calculated flicker correction gain Ga to the flicker correction circuit 22.

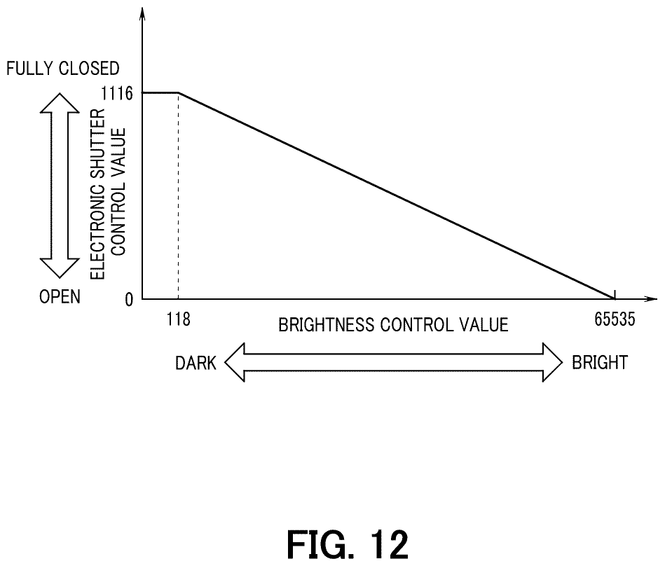

FIG. 12 is a graph showing the relationship between a brightness control value and the electronic shutter control value ES. The brightness control value is a value proportional to the exposure time for controlling the brightness of the image. FIG. 12 shows an example in which the brightness control value is represented by a numerical value of 16-bit (0 to 65535).

As described above, the electronic shutter control value ES is a value representing how many lines of the total number N (N=1120 lines in the above-described example) are in the light-shielded state. Therefore, the less the electronic shutter control value ES, the greater the brightness control value. When the electronic shutter control value ES is the minimum value ESmin=0, the brightness control value is the maximum value of 65535.

As described above, the minimum value n(min) of n is set to 4, for example, according to the specification of the image pickup device 11. In this case, the maximum value ESmax of the electronic shutter control value ES is 1116 for the total number of lines N=1120. The brightness control value at this time is 118, for example.

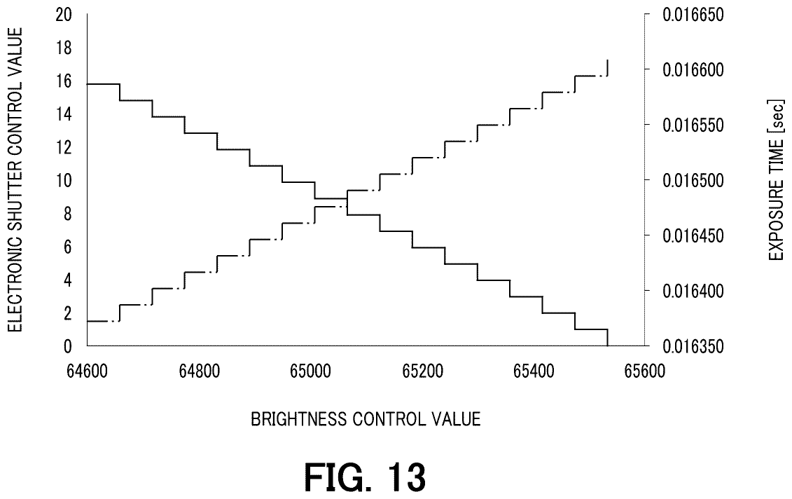

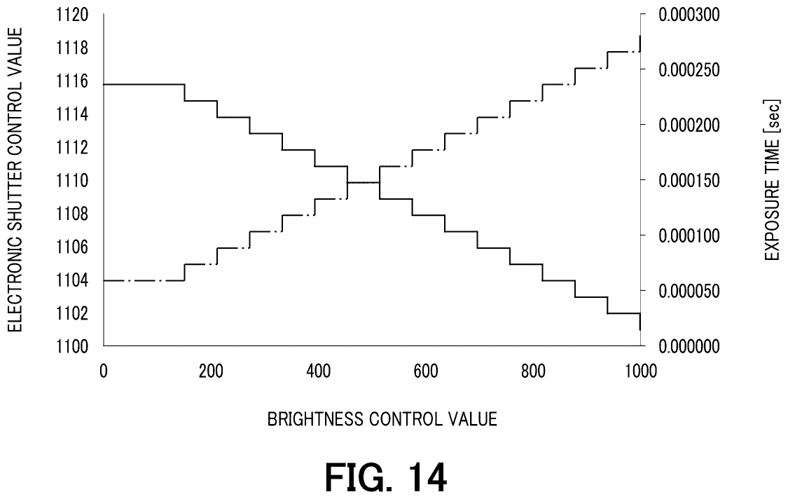

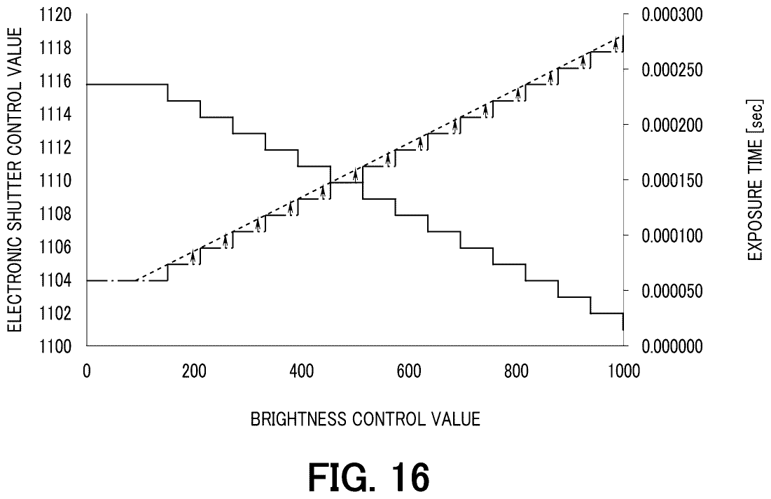

FIG. 13 is a graph showing the vicinity of the maximum value of the brightness control value in FIG. 12 enlarged, along with the exposure time. In the chart of FIG. 13 (and FIG. 14 and FIG. 16 described later), the solid line shows the electronic shutter control value ES, and the dash-dotted line shows the exposure time, respectively.

As described above, the exposure time is controlled by the electronic shutter, for example, on line-by-line. For this reason, the exposure time changes discretely (stepwise).

FIG. 14 is a graph showing the vicinity of the minimum value of the brightness control value in FIG. 12 enlarged, along with the exposure time.

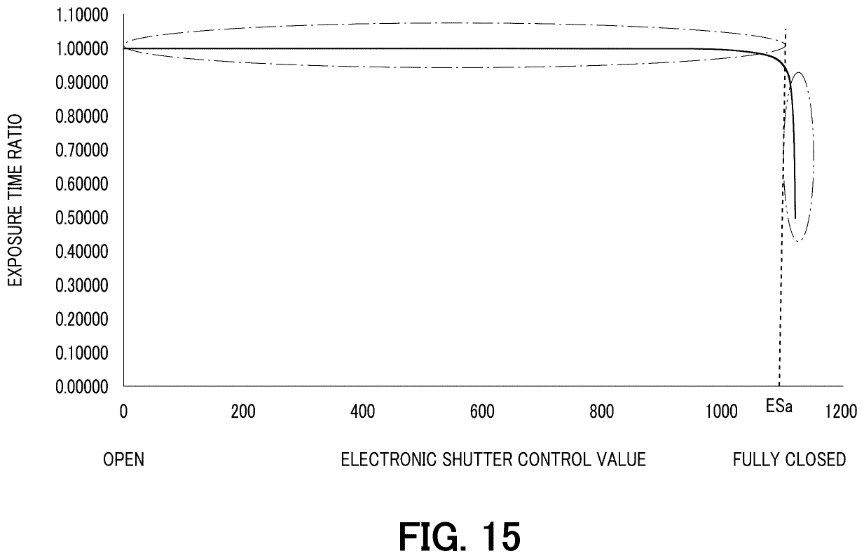

The exposure time changes discretely in the similar manner as in FIG. 13. However, an amount of brightness fluctuation of the image when the electronic shutter control value ES changes by one line differs greatly between the vicinity of the range where the electronic shutter is open shown in FIG. 13 and the vicinity of the range where the electronic shutter is fully closed shown in FIG. 14. This point will be described with reference to FIG. 15.

FIG. 15 is a graph showing a ratio of a change amount of the exposure time when the electronic shutter control value ES changes by one line, to the exposure time. In FIG. 15, the part surrounded by the long dashed double-short dashed line indicates the vicinity of the range where the electronic shutter is fully closed, and the part surrounded by the dash-dotted line indicates other parts.

In the vicinity of the minimum value ESmin of the electronic shutter control value ES, for example, when the electronic shutter control value ES is 120 (lines), the number of lines that are exposed is 1000 (lines). When the electronic shutter control value ES changes by +1 line (or −1 line), the number of lines that are exposed is changed by −1 line (or +1 line), becoming 999 (lines) (or 1001 (lines)). The absolute value of the change ratio of the brightness at this time is 1/1000=0.1(%).

In contrast, in the vicinity of the maximum value ESmax of the electronic shutter control value ES, for example, when the electronic shutter control value ES is 1115 (lines), the number of lines that are exposed is 5 (lines). When the electronic shutter control value ES changes by +1 (or −1) line, the number of lines that are exposed is changed by −1 (or +1) line, becoming 4 (or 6) (lines). The absolute value of the change ratio of the brightness at this time is 1/5=20(%).

Therefore, the fluctuation of the brightness of the image between frames due to discrete control of the exposure time is negligible except in the vicinity of the range where the electronic shutter is fully closed. On the other hand, in the vicinity of the range where the electronic shutter is fully closed, since the brightness of the image greatly fluctuates even when the electronic shutter control value ES changes by 1-line, resulting in the flicker in the image between different frames.

In FIG. 15, the electronic shutter control value ESa indicated by the dotted line is an example of the electronic shutter control value ES that distinguishes whether the flicker in the image due to the discrete control of the exposure time is noticeable. The electronic shutter control value ESa is a value obtained by subtracting “100/target light adjustment accuracy” lines from the total number of lines N. For example, when the total number of lines N is 1120, and the target light adjustment accuracy is 5%, the electronic shutter control value Esa is as follows.

$$ESa = 1120 - 100 / 5 = 1100$$

However, it goes without saying that the above numerical value may be changed as appropriate depending on the total number of lines N, the specification, etc., of the image pickup device 11.

FIG. 16 is a graph showing the vicinity of the minimum value of the brightness control value enlarged and showing how the exposure time that changes discretely is substantially corrected with a gain. In FIG. 16, the dotted line shows the substantial exposure time after the flicker correction.

The flicker correction circuit 22 performs the flicker correction by receiving a flicker correction gain Ga from the flicker correction gain calculation circuit 27, amplifying the image signal received from the first image processing circuit 21 by the flicker correction gain Ga (Step S24).

As in the numerical example described above, the exposure time of the image signal before the flicker correction is shorter by 0.4933%, but by amplifying the image signal with the flicker correction gain Ga=1.004957, the shortage of the exposure time is eliminated.

As a result of the flicker correction circuit 22 performing the image processing of the flicker correction, the stepped exposure time indicated by the dash-dotted line in FIG. 16 is changed to a smooth linear exposure time indicated by the dotted line, and the flicker in the image between frames due to the discrete control of the exposure time is corrected.

According to the first embodiment, an error of the light adjustment control (a quantization error of the exposure time) caused by the discrete control of the exposure time by the electronic shutter is corrected by a gain adjustment of the image processing. As a result, the flicker of the brightness in the image caused in the vicinity of the range where the electronic shutter is fully closed is suppressed, to make the image displayed on the monitor 4 easier to view. Therefore, the diagnosis and treatment by a physician observing the monitor 4 are not hindered by the flicker in the image.

Furthermore, since the gain adjustment is performed using the exposure time ratio er calculated based on the exposure time target value Tgt and the actual exposure time Texp, the fluctuation of the brightness between frames can be suppressed with good accuracy.

Since the flicker in the image in the vicinity of the range where the electronic shutter is fully closed is suppressed, the electronic shutter control value ES corresponding to the range where the electronic shutter is fully closed can be set to a value close to the total number of lines N. This makes it possible to ensure a wide light adjustment dynamic range.

### Second Embodiment

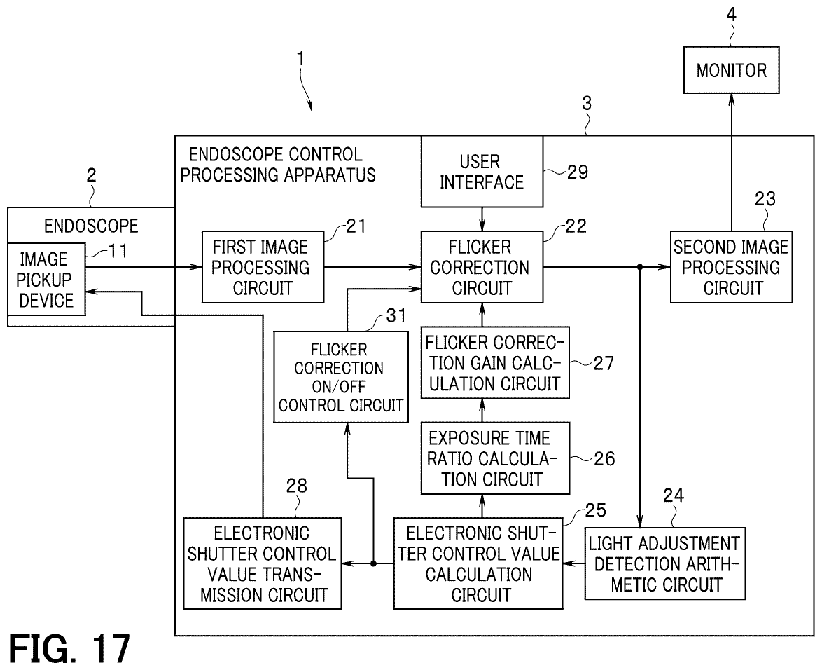

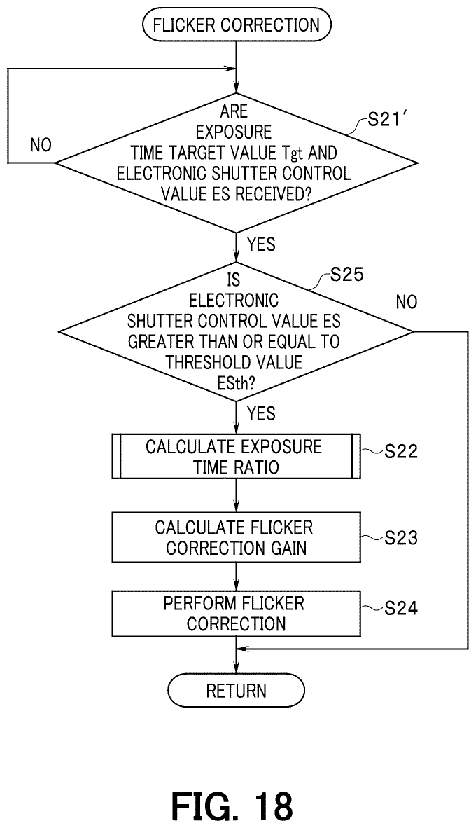

FIG. 17 and FIG. 18 show a second embodiment of the present disclosure. In the second embodiment, the same parts as those in the first embodiment are attached with the same reference signs and the descriptions thereof are omitted, as appropriate. In the second embodiment, points different from the first embodiment will be mainly described.

FIG. 17 is a block diagram showing an electrical configuration of an endoscope system 1.

In an endoscope control processing apparatus 3 of the present embodiment, a flicker correction on/off control circuit 31 is added to the configuration of the first embodiment shown in FIG. 2.

The flicker correction on/off control circuit 31 receives the electronic shutter control value ES from the electronic shutter control value calculation circuit 25, and generates and transmits a control signal to the flicker correction circuit 22.

FIG. 18 is a flowchart showing the processing of the flicker correction in Step S3 of FIG. 5

When the endoscope control processing apparatus 3 enters the processing of the flicker correction in Step S3 of FIG. 5, the processing shown in FIG. 18 is performed.

Then, the exposure time ratio calculation circuit 26 waits to receive the exposure time target value Tgt and the electronic shutter control value ES from the electronic shutter control value calculation circuit 25, and the flicker correction on/off control circuit 31 waits to receive the electronic shutter control value ES from the electronic shutter control value calculation circuit 25 (Step S21′).

When receiving the electronic shutter control value ES, the flicker correction on/off control circuit 31 determines whether the electronic shutter control value ES is greater than or equal to a threshold value ESth (Step S25).

Here, the threshold value ESth is the electronic shutter control value ES that is a guide for distinguishing whether the fluctuation of the brightness in each frame is noticeable as an image flicker due to the error of the light adjustment control caused by the electronic shutter that controls the exposure time discretely. The threshold value ESth is set in advance depending on the specification and the like of the image pickup device 11.

Specifically, the threshold value ESth is set as a value less than or equal to the value obtained by subtracting “100/target light adjustment accuracy” lines from the total number of lines N, as shown in FIG. 15 as the electronic shutter control value ESa.

When the electronic shutter control value ES is greater than or equal to the threshold value ESth, the flicker correction on/off control circuit 31 generates and transmits a flicker correction on-control signal to the flicker correction circuit 22. When receiving the flicker correction on-control signal, the flicker correction circuit 22 performs the flicker correction in Step S24 and returns to the processing of FIG. 5.

In addition, when the electronic shutter control value ES is less than the threshold value ESth, the flicker correction on/off control circuit 31 generates and transmits a flicker correction off-control signal to the flicker correction circuit 22. When receiving the flicker correction off-control signal, the flicker correction circuit 22 does not perform the flicker correction in Step S24, and returns to the processing of FIG. 5.

Therefore, when the electronic shutter control value ES is less than the threshold value ESth, the light adjustment is performed by controlling the electronic shutter using the electronic shutter control value ES calculated by the light adjustment detection, without gain adjustment related to the flicker correction.

Note that when the electronic shutter control value ES is less than the threshold value ESth, only the processing in Step S24 may be skipped after performing the processing in Step S22 and Step S23. However, in the processing of FIG. 18, not only the processing in Step S24 but also the processing in Step S22 and Step S23 are skipped. In this case, the flicker correction on/off control circuit 31 may transmit a processing stop control signal to the exposure time ratio calculation circuit 26 and the flicker correction gain calculation circuit 27. This makes it possible to reduce the power consumption more effectively when the flicker correction is not performed.

As described using the numerical example in the first embodiment, when the number of lines that are exposed is 5 (lines), the absolute value of the change ratio of the brightness when the electronic shutter control value ES is changed by one line is 20(%). On the other hand, when the number of lines that are exposed is 1000 (lines), the absolute value of the change ratio of the brightness is 0.1(%).

Therefore, when the electronic shutter control value ES is less than the threshold value ESth and is far from the range of fully closed, the fluctuation of the brightness in each frame is minute, and the flicker in the image is hardly noticeable.

According to the second embodiment, the same effects as those described above in the first embodiment are achieved. Furthermore, according to the second embodiment, since signal amplification is not performed in scenes where the flicker in the image is not noticeable, a decrease in image quality due to amplification of noise components can be avoided.

### [Additionally Technology 1]

Incidentally, the endoscope control processing apparatus 3 may be configured to serve also as a light source device, for example. FIG. 19 is a view showing a configuration example of the endoscope control processing apparatus 3 serving also as the light source device in an additional technology 1. The additional technology 1 can have some relationship to the other technologies described herein, including that of the embodiments of FIGS. 1-18, and may include some or all of their constituent features, plus the additional features described.

The endoscope 2 includes a light guide 12, an illumination lens 13, an objective lens 14, and a drive circuit 15 in addition to the image pickup device 11 described above.

The light guide 12 transmits illumination light supplied from the endoscope control processing apparatus 3 to the distal end side of the endoscope 2. The light guide 12 is configured, for example, by a fiber bundle consisting of bundled optical fibers, etc.

The illumination lens 13 is provided on the distal end side of the endoscope 2. The illumination lens 13 irradiates a subject with the illumination light transmitted by the light guide 12.

The objective lens 14 forms an optical image of the subject irradiated with the illumination light on the image pickup device 11.

The drive circuit 15 is electrically connected to the image pickup device 11. The drive circuit 15 transmits a driving signal to the image pickup device 11, receives the image signal acquired by the image pickup device 11, and transmits the image signal to the endoscope control processing apparatus 3.

The endoscope control processing apparatus 3 includes a first image processing circuit 21, a second image processing circuit 23, a control circuit 34, a light source drive circuit 35, a light source 36, and a multiplexer 37.

For example, the first image processing circuit 21, the second image processing circuit 23, and the control circuit 34 are composed of the processor 30a and the memory 30b as shown in FIG. 3. However, as described above, a part or all of those may be composed of electronic circuits.

The first image processing circuit 21 performs basic image processing on the image signal received from the endoscope 2.

The second image processing circuit 23 generates an image signal for observing using the monitor 4 from the image signal processed by the first image processing circuit 21, and outputs the image signal to the monitor 4.

The control circuit 34 controls each portion in the endoscope control processing apparatus 3, including the first image processing circuit 21 and the light source drive circuit 35, and also controls the endoscope 2.

The control circuit 34 includes a brightness detection circuit 38 and a light adjustment control circuit 39. The brightness detection circuit 38 receives the image signal from the first image processing circuit 21, performs the light adjustment detection, and detects the brightness of the image. The light adjustment control circuit 39 performs the light adjustment control based on the brightness of the image detected by the brightness detection circuit 38. The light adjustment control by the light adjustment control circuit 39 includes control of the electronic shutter of the image pickup device 11 through the drive circuit 15 and control of the light source 36 through the light source drive circuit 35.

The light source drive circuit 35 controls the light source 36 based on a command from the light adjustment control circuit 39 to cause the light source 36 to emit light.

The light source 36 includes a white light source that emits white illumination light, and further includes, as necessary, a special light source that emits special light. Examples of the special light source includes a laser light source for applying laser light to stones, a light source of excitation light to cause fluorescence from the subject, a light source for performing narrow band imaging (NBI), etc.

Specifically, the light source 36 includes a plurality of light-emitting elements, such as light emitting diodes (LEDs), which emit light of different wavelengths. For example, the LEDs that constitute the white light source include white LEDs. Alternatively, the LEDs constituting the white light source may be configured to include LEDs that emit green (G) light, LEDs that emit red (R) light, and LEDs that emit blue (B) light. The special light source includes light-emitting elements such as LEDs that emit light of other wavelength and laser diodes (LDs).

The light source 36 emits light from one or more light-emitting elements under control of the light source drive circuit 35.

The multiplexer 37 multiplexes a plurality of light with different wavelengths emitted from the light source 36, and supplies the plurality of light as illumination light to the endoscope 2.

Note that the light source device may be provided separately from the endoscope control processing apparatus 3.

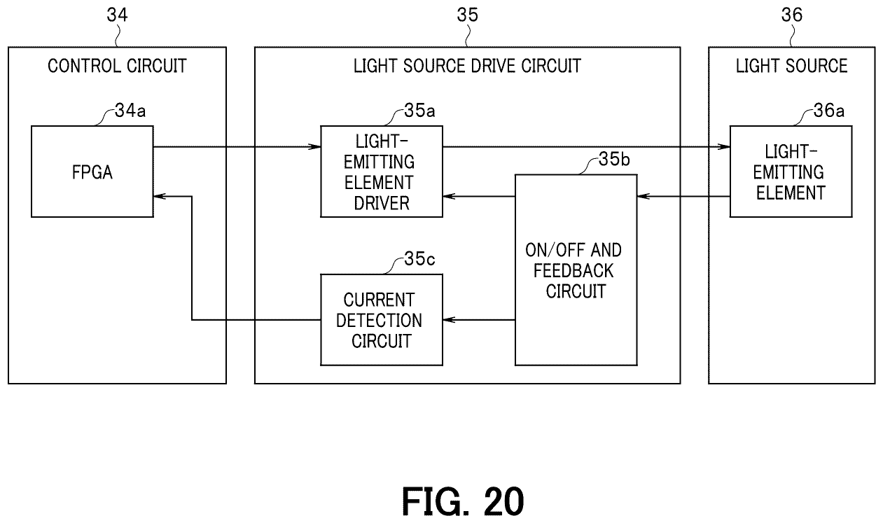

FIG. 20 is a block diagram showing a configuration example of the control circuit 34, the light source drive circuit 35, and the light source 36 in the additional technology 1.

The control circuit 34 is configured to include, for example, an FPGA 34a as the processor 30a.

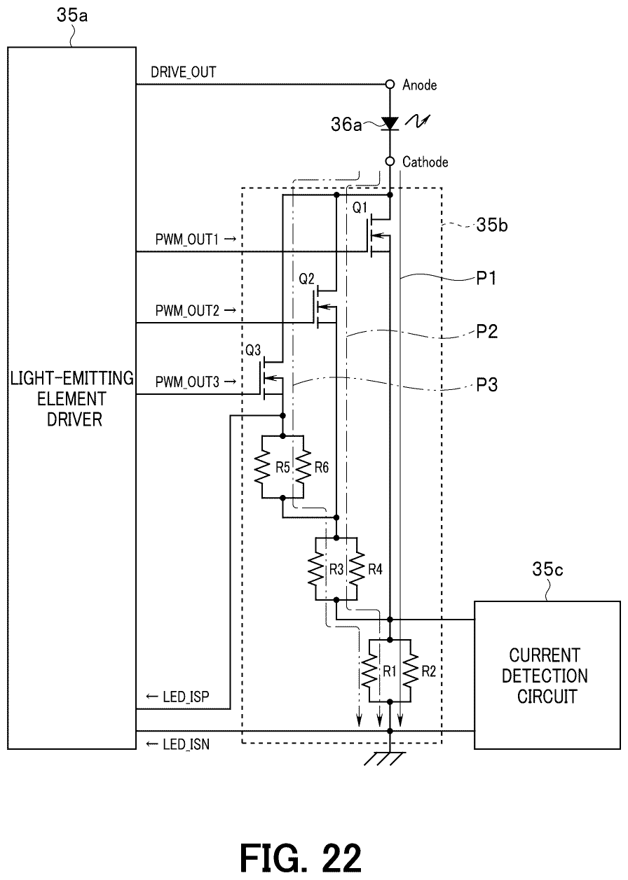

The light source drive circuit 35 includes a light-emitting element driver 35a, an on/off and feedback circuit 35b, and a current detection circuit 35c. A more specific configuration of the light source drive circuit 35 will be described later with reference to FIG. 22.

The light source 36 includes a light-emitting element 36a.

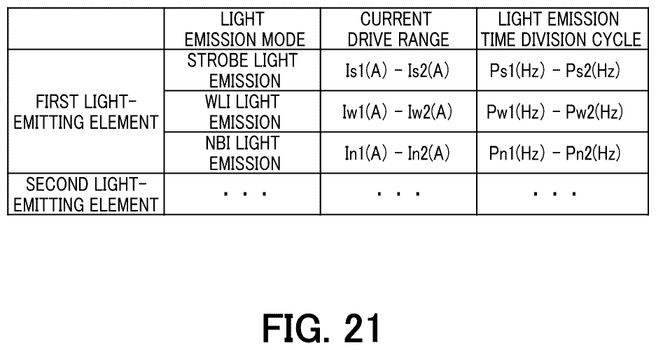

FIG. 21 is a diagram showing an example of a light emission mode of the endoscope control processing apparatus 3 serving also as the light source device in the additional technology 1.

The endoscope control processing apparatus 3 emits illumination light in a plurality types of light emission modes. In the example shown in FIG. 21, the endoscope control processing apparatus 3 can be set to a strobe light emission mode, a white light imaging (WLI) light emission mode, and an NBI light emission mode as the light emission mode of the illumination light.

As described above, the light-emitting element 36a includes a plurality of light-emitting elements (a first light-emitting element, a second light-emitting element, etc.). For example, focusing on the first light-emitting element, as shown in FIG. 21, the first light-emitting element is driven with different current drive ranges and different light emission time division cycles in respective the three light emission modes.

Furthermore, different light-emitting elements, such as the first light-emitting element and the second light-emitting element have the different current drive ranges and the different light emission time division cycles in the three light emission modes.

FIG. 22 is a view showing a more specific configuration example of the light source drive circuit 35 and the light source 36 in the additional technology 1.

The light-emitting element driver 35a is configured as an integrated circuit (IC), for example, and includes a plurality of output terminals and a plurality of input terminals.

A drive output terminal DRIVE_OUT of the light-emitting element driver 35a is connected to an anode of the light-emitting element 36a configured as an LED, for example. A cathode of the light-emitting element 36a is connected to the on/off and feedback circuit 35b.

The on/off and feedback circuit 35b includes three transistors Q1 to Q3 that function as switches and six resistors R1 to R6.

The transistors Q1 to Q3 are each configured as an enhancement-type and N-channel power metal-oxide-semiconductor field-effect transistor (MOSFET), for example. The enhancement-type MOSFET does not allow drain current to flow when a gate-source voltage is 0.

The transistors Q1 to Q3 are arranged in parallel with each other, with each drain connected to the cathode of the light-emitting element 36a. Agate of the transistor Q1 is connected to a control output terminal PWM_OUT1 of the light-emitting element driver 35a, a gate of the transistor Q2 is connected to a control output terminal PWM_OUT2 of the light-emitting element driver 35a, and a gate of the transistor Q3 is connected to a control output terminal PWM_OUT3 of the light-emitting element driver 35a, respectively.

A source of the transistor Q1 is connected to one ends of the resistors R1 and R2 which are arranged in parallel. The other ends of the resistors R1 and R2 are connected to the ground.

A source of the transistor Q2 is connected to one ends of the resistors R3 and R4, which are arranged in parallel. The other ends of the resistors R3 and R4 are connected to the source of the transistor Q1 and the one ends of the resistors R1 and R2.

A source of the transistor Q3 is connected to one ends of the resistors R5 and R6, which are arranged in parallel. The other ends of the resistors R5 and R6 are connected to the source of the transistor Q2 and the one ends of the resistors R3 and R4.

Note that arranging the plurality of resistors are arranged in parallel has an advantage of lowering the temperature of each resistor and providing heat countermeasures.

A positive side input terminal LED_ISP of the light-emitting element driver 35a is connected between the source of the transistor Q3 and the one ends of the resistors R5 and R6.

The current detection circuit 35c is connected to both ends of the resistors R1 and R2 which are arranged in parallel. A negative side input terminal LED_ISN of the light-emitting element driver 35a is connected to other ends of the resistors R1 and R2. The light-emitting element driver 35a performs feedback control of each output based on inputs to the positive side input terminal LED_ISP and the negative side input terminal LED_ISN.

In the configuration shown in FIG. 22, when the light-emitting element 36a is caused to emit light, the three transistors Q1 to Q3 are selectively controlled so that only one of those is turned on.

In the strobe light emission mode, the transistor Q1 is controlled to be on, and the transistors Q2 and Q3 are controlled to be off. A current path in the strobe light emission mode is indicated as P1 by a solid line. The light-emitting element driver 35a controls a current value in the current path P1 and controls a time during which the transistor Q1 is turned on (current flow time), thereby controlling the light emission amount in the strobe light emission mode.

Furthermore, in the WLI light emission mode, the transistor Q2 is controlled to be on, and the transistors Q1 and Q3 are controlled to be off. A current path in the WLI light emission mode is indicated as P2 by a dash-dotted line. At this time, the light-emitting element driver 35a controls a current value of the current path P2, and performs pulse-width modulation (PWM) control on the transistor Q2 to control the light emission amount in the WLI light emission mode.

In the NBI light emission mode, the transistor Q3 is controlled to be on, and the transistors Q1 and Q2 are controlled to be off. A current path in the NBI light emission mode is indicated as P3 by a long dashed double-short dashed line. At this time, the light-emitting element driver 35a controls a current value of the current path P3, and performs the PWM control on the transistor Q3 to control the light emission amount in the NBI light emission mode.

It is supposed that that current in the current path P1 is I1, and an input voltage of the current detection circuit 35c in the strobe light emission mode is V1. It is supposed that that current in the current path P2 is I2, and an input voltage of the current detection circuit 35c in the WLI light emission mode is V2. It is supposed that current in the current path P3 is I3, and an input voltage of the current detection circuit 35c in the NBI light emission mode is V3. Here, it is supposed that a minimum value and a maximum value in a drive range of each current are denoted by adding min and max, respectively. In addition, resistance values of the resistors R1 to R6 are denoted by using the same symbols R1 to R6. Furthermore, a combined resistance value when two resistors are connected in parallel is denoted by the symbol “//”.

Specifically, combined resistance value (R1//R2), (R3//R4), (R5//R6) are the values shown in Equations 6 to 8 below, respectively.

$$( R 1 // R 2 ) = R 1 \times R 2 / ( R 1 + R 2 ) \qquad ( Equation 6 )$$

$$( R 3 // R 4 ) = R 3 \times R 4 / ( R 3 + R 4 ) \qquad ( Equation 7 )$$

$$( R 5 // R 6 ) = R 5 \times R 6 / ( R 5 + R 6 ) \qquad ( Equation 8 )$$

The current detection circuit 35c detects a current value from voltage values at both ends of the combined resistor (R1//R2). Then, each of the ranges of the voltages V1, V2, and V3 detected by the current detection circuit 35c as being within the normal range are given by the following Equations 9 to 11.

$$I 1 min \times ( R 1 // R 2 ) < V 1 < I 1 max \times ( R 1 // R 2 ) \qquad ( Equation 9 )$$

$$I 2 min \times \frac{( R 1 // R 2 )}{( R 1 // R 2 ) + ( R 3 // R 4 )} < V 2 < I 2 max \times \frac{( R 1 // R 2 )}{( R 1 // R 2 ) + ( R 3 // R 4 )} \qquad ( Equation 10 )$$

$$I 3 min \times \frac{( R 1 // R 2 )}{( R 1 // R 2 ) + ( R 3 // R 4 ) + ( R 5 // R 6 )} < V 3 < I 3 max \times \frac{( R 1 // R 2 )}{( R 1 // R 2 ) + ( R 3 // R 4 ) + ( R 5 // R 6 )} \qquad ( Equation 11 )$$

In this way, the different current paths P1 to P3 are provided for each light emission mode, and the current paths P1 to P3 are combined to detect the current. At this time, by appropriately designing each combined resistance value (R1//R2), (R3//R4), and (R5//R6) to correspond to the current drive range in each light emission mode, it is possible to detect the current in each light emission mode with the one current detection circuit 35c.

A generally conceivable configuration is one in which a plurality of current detection circuits are provided for a plurality of current paths, respectively. In this case, the current detection circuit can be designed according to the current drive range for each light emission mode, but one current detection circuit is required for each current path.

Compared to the general configuration in which the current detection circuit is provided for each light emission mode, the configuration shown in FIG. 22 allows for a simpler circuit configuration because only one current detection circuit 35c is required.

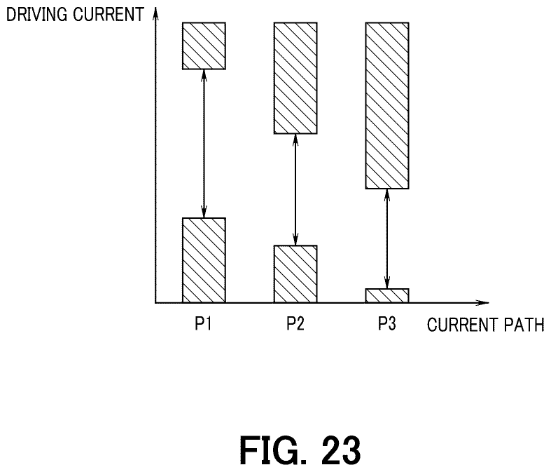

FIG. 23 is a graph showing an example of ranges in which driving current detected by the current detection circuit 35c in each light emission mode is normal and abnormal in the additional technology 1.

In FIG. 23, current values corresponding to voltages in the normal range shown in the diagram of the FIG. 21 and Equation 9 to Equation 11 are indicated by arrows, and current values corresponding to voltages in the abnormal range are indicated by hatched bar charts. In the illustrated example, the median value of the current values in the normal range decreases in the order of the strobe light emission mode (the current path P1), the WLI light emission mode (the current path P2), and the NBI light emission mode (the current path P3), for example.

The FPGA 34a of the control circuit 34 receives the current value detected by the current detection circuit 35c, and determines whether the received current value is in the normal range or the abnormal range in the current light emission mode.

For example, when operating in the WLI light emission mode, if a component fails and the light-emitting element 36a is driven in the strobe light emission mode, strong light may be emitted onto the subject. The FPGA 34a compares the current value detected by the current detection circuit 35c with the normal driving current range, and detects an abnormal state such as operation in an unintended light emission mode. When detecting the abnormal state, the FPGA 34a controls the light-emitting element driver 35a to stop light emission by turning off all the transistors Q1 to Q3, for example.

Note that it is not limited to this, when detecting the abnormal state, the FPGA 34a may control to switch to other safe drive state (or safe drive path). For example, when detecting the abnormal state, the FPGA 34a may switch to the NBI light emission mode, which has the lowest median value in the normal range of the current value among the three light emission modes, or the like.

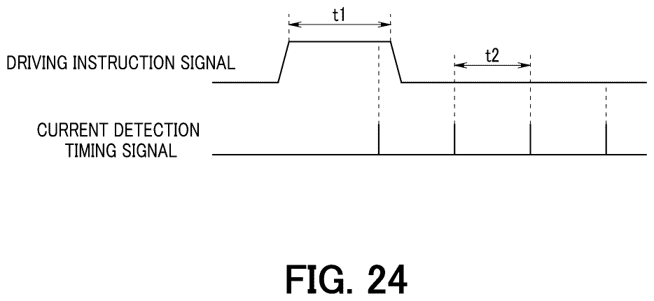

FIG. 24 is a timing chart showing an example of timing at which the current detection circuit 35c performs current detection in the additional technology 1.

The current detection circuit 35c receives a current detection timing signal transmitted from the control circuit 34, for example, and performs the current detection at the received timing. For example, the current detection timing signal is a pulse signal that becomes a high level at a fixed cycle t2. The current detection circuit 35c performs the current detection when the pulse signal becomes the high level.

A driving instruction signal shown in FIG. 24 is a signal outputted from each of the control output terminals PWM_OUT1 to PWM_OUT3 of the light-emitting element driver 35a. A period during which the driving instruction signal is at a high level and one of the transistors Q1 to Q3 is turned on is t1. Then, the cycle t2 of the current detection timing signal is set so that the cycle t2 is shorter than the period t1 (t1>t2). With this, the current detection circuit 35c certainly performs the current detection at least once during the period t1 during which one of the transistors Q1 to Q3 is turned on.

By performing the current detection during the period t1, it is possible to confirm whether the light-emitting element 36a is being driven with the intended current. For example, in the WLI light emission mode, it is possible to confirm that the light-emitting element 36a is not operated within the current drive range of the strobe light emission mode.

Furthermore, by performing the current detection in a period other than the period t1, it is possible to confirm whether or not the current is flowing thorough the light-emitting element 36a at a timing when the current drive is not instructed, i.e., at an unintended timing. For example, in the strobe light emission mode, it is possible to confirm that the light-emitting element 36a is not emitting light at a turn-off timing for strobe light emission.

Note that since the period t1 differs depending on the light emission mode, the cycle t2 is also set according to the light emission mode. Alternatively, the cycle t2 may be set shorter than the period t1 in any light emission mode.

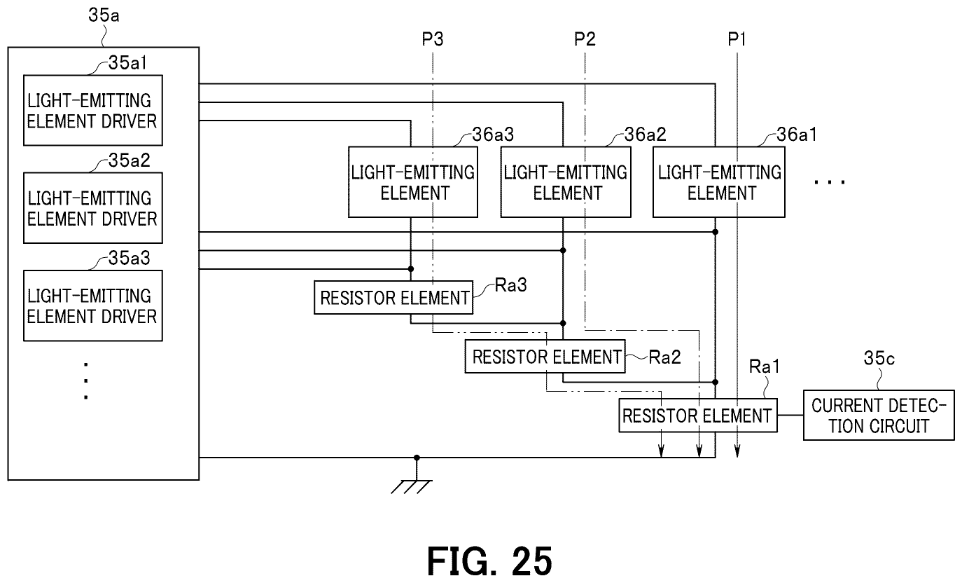

FIG. 25 is a view showing an example in which one current detection circuit 35c is applied to a configuration in which a plurality of light-emitting elements 36a1, 36a2, 36a3, . . . are driven by a plurality of light-emitting element drivers 35a1, 35a2, 35a3, . . . respectively in the additional technology 1.

The light-emitting element driver 35a includes the plurality of light-emitting element drivers 35a1, 35a2, 35a3, . . . . Furthermore, on the current paths P1 to P3, resistor elements Ra1, Ra2, Ra3, . . . are disposed as described below.

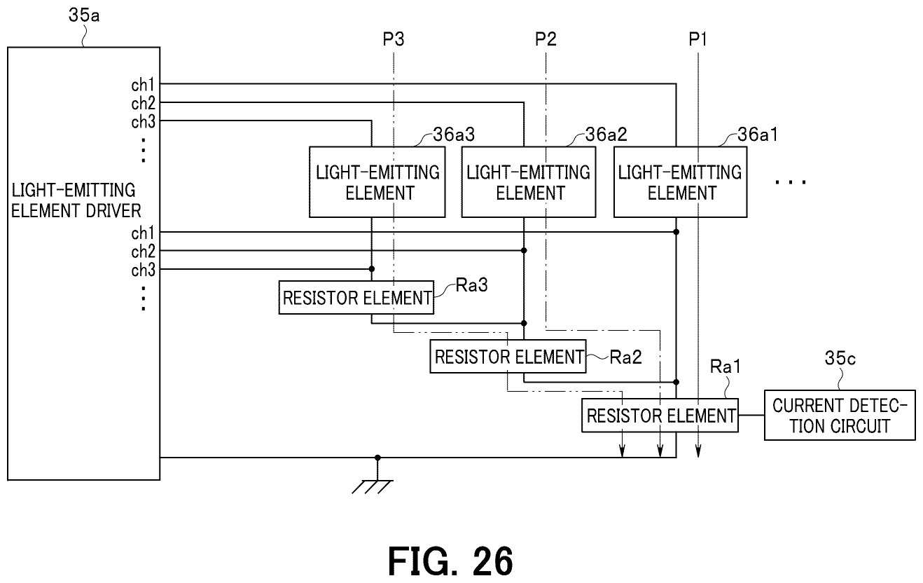

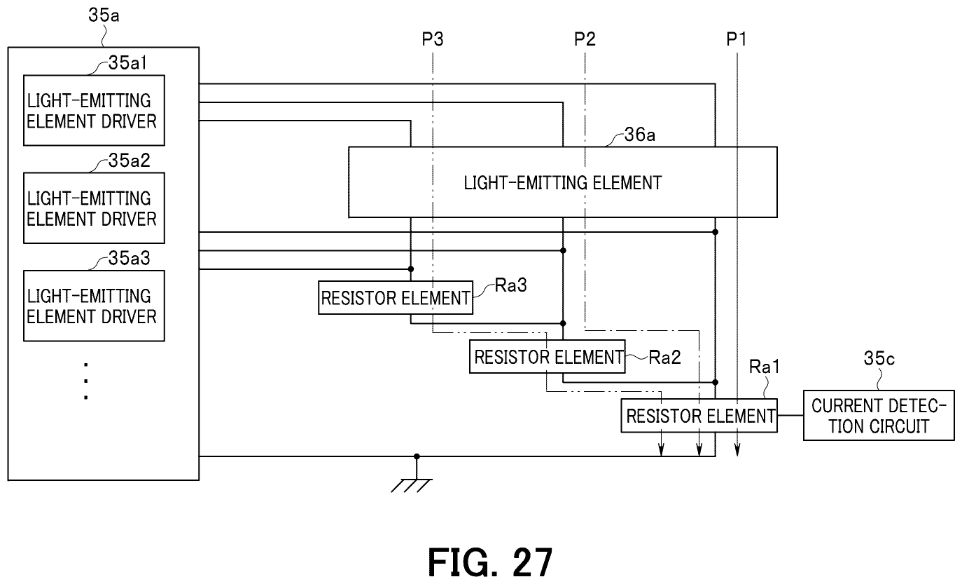

Note that the numbers of the light-emitting elements, the light-emitting element drivers, and the resistor elements may be two, or more than three, but the following describes an example in which the numbers are three. Note that FIG. 26 and FIG. 27 shown below will be described according to the example in which the numbers are three.

In a first light emission mode (for example, the strobe light emission mode), the light-emitting element 36a1 is driven by the light-emitting element driver 35a1, and the current path P1 passes through the light-emitting element 36a1 and the resistor element Ra1.

In a second light emission mode (for example, the WLI light emission mode), the light-emitting element 36a2 is driven by the light-emitting element driver 35a2, and the current path P2 passes through the light-emitting element 36a2 and the resistor elements Ra2 and Ra1.

In a third light emission mode (for example, the NBI light emission mode), the light-emitting element 36a3 is driven by the light-emitting element driver 35a3, and the current path P3 passes through the light-emitting element 36a3 and the resistor elements Ra3, Ra2, and Ra1. Note that the current paths P1 to P3 are selectively used in the same manner as described above.

The current paths P1 to P3 converge at the resistor element Ra1. The current detection circuit 35c is arranged to measure a current value based on a voltage of the resistor element Ra1.

Therefore, in the configuration of FIG. 25, by appropriately setting each of the resistance values of the resistor elements Ra1, Ra2, and Ra3, it is possible to detect the current values flowing through the plurality of current paths P1 to P3 with the one current detection circuit 35c.

FIG. 26 is a view showing an example in which the one current detection circuit 35c is applied to a configuration in which the plurality of light-emitting elements 36a1, 36a2, and 36a3 are driven by one light-emitting element driver 35a in the additional technology 1.

In the configuration of FIG. 26 compared to the configuration of FIG. 25, the plurality of light-emitting elements 36a1, 36a2, and 36a3 are respectively driven by different channels ch1, ch2, ch3, . . . of the one light-emitting element driver 35a. The channel ch1 is connected to the light-emitting element 36a1, the channel ch2 is connected to the light-emitting element 36a2, and the channel ch3 is connected to the light-emitting element 36a3.

The configurations of the light-emitting elements 36a1, 36a2, and 36a3, the resistor elements Ra1, Ra2, and Ra3, and the current detection circuit 35c are basically the same as those shown in FIG. 25. In addition, the current paths P1 to P3 are selectively used in the same manner as described above.

For example, the first light emission mode (for example, the strobe light emission mode) is controlled by the channel ch1, the second light emission mode (for example, the WLI light emission mode) is controlled by the channel ch2, and the third light emission mode (for example, the NBI light emission mode) is controlled by the channel ch3.

The current paths P1 to P3 converge at the resistor element Ra1. The current detection circuit 35c is arranged to measure a current value based on the voltage of the resistor element Ra1.

Therefore, even in the configuration of FIG. 26, it is possible to detect the current values flowing through the plurality of current paths P1 to P3 with the one current detection circuit 35c, as in the configuration of FIG. 25.

FIG. 27 is a diagram showing an example of applying the one current detection circuit 35c to a configuration in which the one light-emitting element 36a is driven by the plurality of light-emitting element drivers 35a1, 35a2, and 35a3 in the additional technology 1.

The plurality of light-emitting element drivers 35a1, 35a2, and 35a3 drive the light-emitting element 36a in the different light emission modes respectively, for example.

In the first light emission mode (for example, the strobe light emission mode), the light-emitting element 36a is driven by the light-emitting element driver 35a1, and the current path P1 passes through the light-emitting element 36a and the resistor element Ra1.

In the second light emission mode (for example, the WLI light emission mode), the light-emitting element 36a is driven by the light-emitting element driver 35a2, and the current path P2 passes through the light-emitting element 36a and the resistor elements Ra2 and Ra1.

In the third light emission mode (for example, the NBI light emission mode), the light-emitting element 36a is driven by the light-emitting element driver 35a3, and the current path P3 passes through the light-emitting element 36a and the resistor elements Ra3, Ra2, and Ra1. Note that the current paths P1 to P3 are selectively used as described above.

The current paths P1 to P3 converge at the resistor element Ra1. The current detection circuit 35c is arranged to measure a current value based on the voltage of the resistor element Ra1.

Therefore, also in the configuration of FIG. 27, it is possible to detect the current values flowing through the plurality of current paths P1 to P3 by the one current detection circuit 35c as in the configurations of FIG. 25 and FIG. 26.

According to the additional technology 1 shown in FIG. 19 to FIG. 27, there are a plurality of current paths passing through the light-emitting element 36a, and the circuit elements or the paths through which the current flows in common are provided in the light source drive circuit 35 having the different current drive ranges for each current path, even if the light-emitting element 36a is driven in any of the current paths. Then the current detection is performed by the one current detection circuit 35c on the circuit elements or the paths.

As a result, even in a case where the light-emitting element 36a is driven in any of the current paths, the current can be detected by the one current detection circuit 35c. Therefore, abnormal current at the time of failure can be detected collectively by the one current detection circuit 35c, thereby preventing malfunction of the light source device.

When detecting the abnormal state, the FPGA 34a switches the light source drive circuit 35 and the light source 36 to a safe drive state (or safe drive path). With this, it is possible to certainly prevent from the subject being continuously irradiated with the illumination light in the abnormal state. Therefore, the subject is not continuously exposed to strong light, for example.

### [Additional Technology 2]

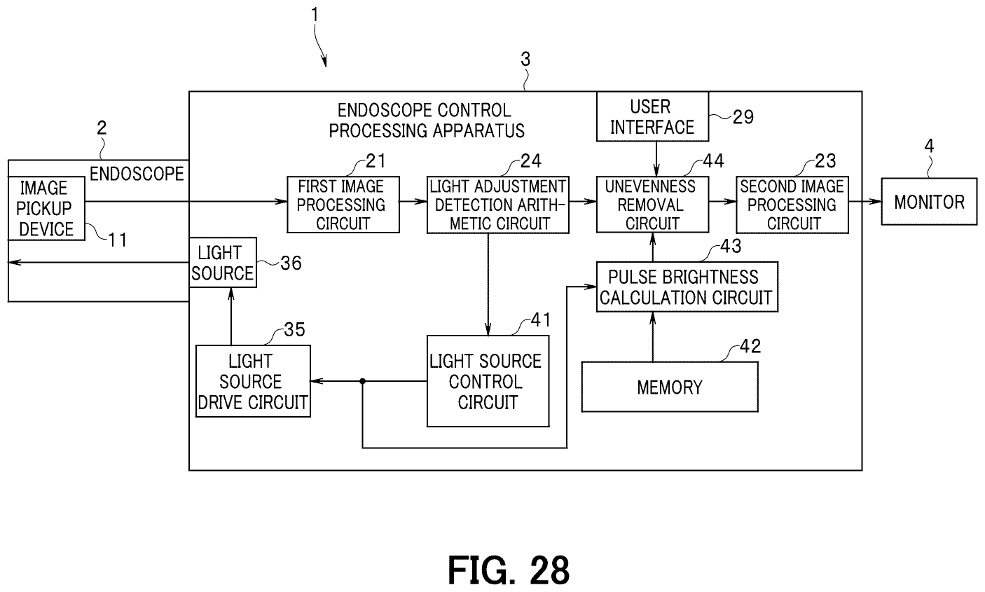

FIG. 28 is a view showing a configuration example of an endoscope control processing apparatus 3 serving also as a light source device in an additional technology 2. The additional technology 2 can have some relationship to the other technologies described herein, including that of the embodiments of FIGS. 1-18, and/or additional technology 1, and may include some or all of their constituent features, plus the additional features described.

In the endoscope, observation is performed in the strobe light emission mode in which strobe light emission is performed in synchronization with a periodic vibration of the subject (a strobe observation mode). An example of an observation target by the strobe observation mode is vocal cords that vibrate periodically. Other examples of the observation target by the strobe observation mode include a heart that beats periodically and arteries that pulse periodically.

In such strobe observation, unevenness removal processing is performed for the purpose of correcting exposure unevenness between frames or lines.

For example, Japanese Patent Application Laid-Open Publication No. 2020-151402 describes that unevenness removal processing is performed based on the wave crest value of LED current when controlling the LED current to perform light adjustment in the strobe observation mode. In other words, in this publication, the wave crest value (current value) of the LED current is used as brightness information for each pulsed light.

However, LEDs generally do not have a completely linear (directly proportional) relationship between current and brightness. Therefore, using the wave crest value of the LED current as it is as the brightness information for each pulsed light results in errors between the brightness based on the wave crest value and the actual brightness, thereby reducing the accuracy of the unevenness removal processing.

Therefore, FIG. 28 shows a configuration example that improves the accuracy of the unevenness removal processing.

An endoscope system 1 includes an endoscope 2, the endoscope control processing apparatus 3, and a monitor 4 as described above.

The endoscope 2 is configured as an electronic endoscope as described above and includes an image pickup device 11.

The endoscope control processing apparatus 3 includes the first image processing circuit 21, the second image processing circuit 23, the light adjustment detection arithmetic circuit 24, the user interface 29, the light source drive circuit 35, and the light source 36 that are described above. The endoscope control processing apparatus 3 further includes a light source control circuit 41, a memory 42, a pulse brightness calculation circuit 43, and an unevenness removal circuit 44.

The light source 36 includes, for example, the light-emitting element 36a (see FIG. 20, etc.) configured as an LED, as described above.

The light source control circuit 41 acquires a light adjustment detection value from the light adjustment detection arithmetic circuit 24. The light source control circuit 41 generates a signal for controlling the light source 36 through the light source drive circuit 35, based on the light adjustment detection value.

The signal generated by the light source control circuit 41 includes a PWM control signal and a current value control signal, for example. The PWM control signal in the strobe light emission mode is a signal in synchronization with the periodic vibration of the subject. The light source drive circuit 35 supplies rectangular pulse-shaped current to the light source 36 at the timing indicated by the PWM control signal. The light source 36 emits pulsed light when the current is supplied.

The memory 42 functions as a conversion parameter storage circuit. The memory 42 stores conversion parameters for converting a current control value I indicated by the current value control signal into pulse brightness L in a non-volatile manner. The memory 42 may be a part of the memory 30b shown in FIG. 3, or may be provided separately from the memory 30b.

The current value control signal generated by the light source control circuit 41 is also transmitted to the pulse brightness calculation circuit 43. The pulse brightness calculation circuit 43 reads the conversion parameters from the memory 42, and converts the current control value I indicated by the current value control signal into the pulse brightness L.

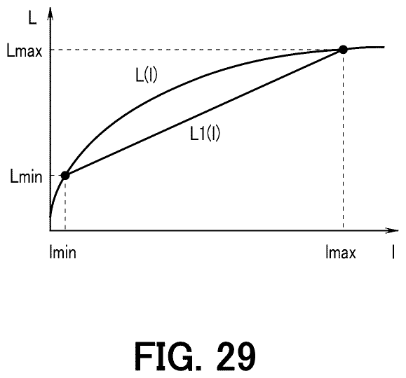

FIG. 29 is a graph showing a first example of the conversion parameters for converting the current control value I into the pulse brightness L in the additional technology 2.

As indicated by the curved line L(I) in FIG. 29, the current control value I and the pulse brightness L generally do not have a linear relationship. The maximum value of the current control value I is denoted as Imax, and the maximum value of the pulse brightness when I=Imax is denoted as Lmax=L(Imax). At this time, assuming linearity by connecting (I, L)=(0, 0) and (I, L)=(Imax, Lmax) with a straight line, calculating the pulse brightness L from the current control value I results in a large error between the calculated pulse brightness L and the actual pulse brightness.

Therefore, as indicated by the straight line L1(I) in FIG. 29, a straight line closer to the curved line L(I) is set. The minimum value of the current control value I is denoted as Imin, and the minimum value of the pulse brightness when I=Imin is denoted as Lmin=L (Imin). The straight line L1(I) is set as a straight line connecting (I, L)=(Imin, Lmin) and (I, L)=(Imax, Lmax). The straight line L1(I) is expressed by the linear function formula shown in the following Equation 12 using an inclination k1 and an intercept m1.

$$L 1 ( I ) = k 1 \times I + m 1 \qquad ( Equation 12 )$$

As can be seen in FIG. 29, even if the straight line L1(I) is extended, it generally does not pass through (I, L)=(0, 0). It can be seen that the straight line L1(I) has a smaller error with the curved line L(I) indicating the actual pulse brightness than the straight line passing through (I, L)=(0, 0).

The memory 42 stores the inclination k1 and the intercept m1, or (Imin, Lmin) and (Imax, Lmax) as the conversion parameters.

The pulse brightness calculation circuit 43 reads, for example, the inclination k1 and the intercept m1 from the memory 42, and converts the current control value I indicated by the current value control signal into information of the pulse brightness L based on Equation 12. However, instead of this, the pulse brightness calculation circuit 43 may read (Imin, Lmin) and (Imax, Lmax) from the memory 42, and calculate the pulse brightness L corresponding to the current control value I by interpolation arithmetic or the like.

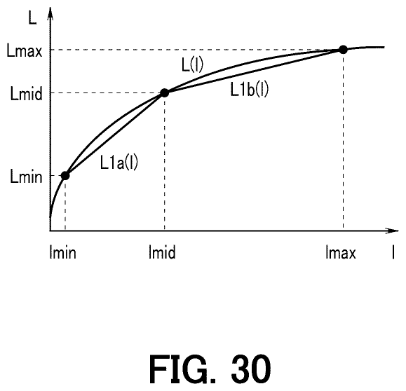

FIG. 30 is a graph showing a second example of the conversion parameters for converting the current control value I into the pulse brightness L in the additional technology 2.

In FIG. 29, the straight line L(I) connecting the minimum value point (Imin, Lmin) and the maximum value point (Imax, Lmax) is obtained, and FIG. 30 is the example in which the intermediate value point (Imid, Lmid) is further added to approximate the curved line L(I) with a broken line.

The intermediate value point (Imid, Lmid) may be set, for example, so that the value in which the area of a part enclosed by the curved line L(I) and the straight line L1a(I) and the area of a part enclosed by the curved line L(I) and the straight line L1b(I) are added becomes the minimum value.

Alternatively, the point (Imid, Lmid) may be set such that Imid=(Imin+Imax)/2 and Lmid=L(Imid). Furthermore, the point (Imid, Lmid) may be set such that Lmid=(Lmin+Lmax)/2 and Imid=L\hat{ }(−1)(Lmid). Here, L\hat{ }(−1)(L) represents the inverse function of curved line L(I).

The straight line L1a(1) is set as the straight line connecting (I, L)=(Imin, Lmin) and (I, L)=(Imid, Lmid). The straight line L1a(I) is expressed by the linear function formula shown in the following Equation 13 using the inclination k1a and the intercept m1a.

$$L 1 a ( I ) = k 1 a \times I + m 1 a \qquad ( Equation 13 )$$

The straight line L1b(I) is set as the straight line connecting (I, L)=(Imid, Lmid) and (I, L)=(Imax, Lmax). The straight line L1b(I) is expressed by the linear function formula shown in the following Equation 14 using the inclination k1b and the intercept m1b.

$$L 1 b ( I ) = k 1 b \times I + m 1 b \qquad ( Equation 14 )$$

As can be seen by comparing FIG. 30 with FIG. 29, the broken line consisting of the straight line L1a(I) and the straight line L1b(I) has a smaller error with respect to the curved line L(I) indicating the actual pulse brightness than the straight line L1(I).

The memory 42 stores the inclination k1a and the intercept m1a, or (Imin, Lmin) and (Imid, Lmid) as first conversion parameters. In addition, the memory 42 stores the inclination k1b and the intercept m1b, or (Imid, Lmid) and (Imax, Lmax) as second conversion parameters.

The pulse brightness calculation circuit 43 reads, for example, the inclination k1a and the intercept m1a from the memory 42 when the current control value I is less than Imid, and converts the current control value I into the information of the pulse brightness L based on Equation 13. However, instead of this, the pulse brightness calculation circuit 43 may read (Imin, Lmin) and (Imid, Lmid) from the memory 42 and calculate the pulse brightness L corresponding to the current control value I by interpolation arithmetic or the like.

On the other hand, when the current control value I is greater than or equal to Imid, the pulse brightness calculation circuit 43 reads, for example, the inclination k1b and the intercept m1b from the memory 42, and converts the current control value I into the information of the pulse brightness L based on Equation 14. However, instead of this, the pulse brightness calculation circuit 43 may read (Imid, Lmid) and (Imax, Lmax) from the memory 42, and calculate the pulse brightness L corresponding to the current control value I by interpolation arithmetic or the like.

Note that the broken line is formed by three points in FIG. 30, but may be formed by four or more points. In this case, the number of types of the linear function formulas calculated is “the number of acquired data points −1”.

The conversion parameters as shown in FIG. 29 or FIG. 30 are required in a process inspection. In other words, in the process inspection, the relationship between the current control value I and the pulse brightness L is obtained for two or more points including the minimum value point (Imin, Lmin) and the maximum value point (Imax, Lmax). Then, the conversion parameters are calculated based on the straight line connecting the two points or the broken line connecting the three or more points.

Note that the process inspection may be performed on each individual endoscope control processing apparatus 3. In addition, results of the process inspection performed on a representative individual of the endoscope control processing apparatus 3 may be applied to respective individuals of the same product.

In the above description, the linear function formula is used, but it is not limited this, and a quadratic or higher degree function formula may be used.

In addition, in the above description, the pulse brightness calculation circuit 43 reads and uses the conversion parameters stored in the memory 42, but it is not limited to this. For example, if the pulse brightness calculation circuit 43 preconfigured with an arithmetic circuit corresponding to the above described function formula is used, the memory 42 is not necessary. For example, when the conversion parameters for each individual are used, the memory 42 may be used. In addition, if the conversion parameters common to each individual of the same product, either a configuration with or without the memory 42 may be adopted.

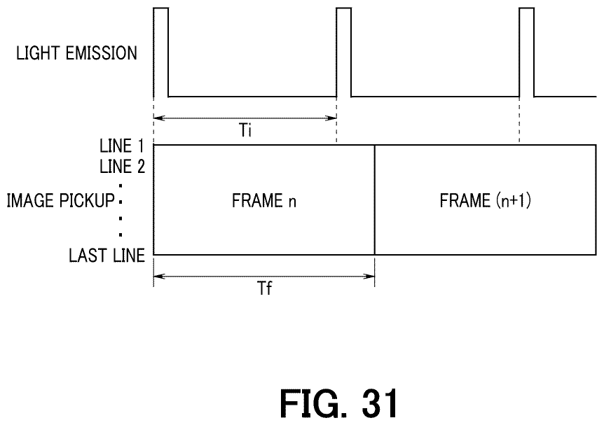

FIG. 31 is a timing chart for explaining exposure unevenness between frames when the image pickup device 11 is a global shutter type in the additional technology 2.

As described above, the emission of the pulsed light by the light source 36 is performed in synchronization with the vibration of the subject (for example, the vibration of the vocal cords as described above). Therefore, the pulse light emission of the light source 36 is generally not synchronized with the frame rate.

When the image pickup device 11 is a global shutter type such as a CCD imager, for example, the number of times of the pulse light emission in the exposure period of one frame may not be the same between different frames.

FIG. 31 shows an example in which the light emission cycle Ti is shorter than a frame cycle Tf, that is, Ti<Tf. The pulse light emission is performed twice within the exposure period of the frame n, but pulse light emission is performed only once in the exposure period of the frame (n+1), resulting in the exposure unevenness between the frames. The exposure unevenness between frames is also called flicker.

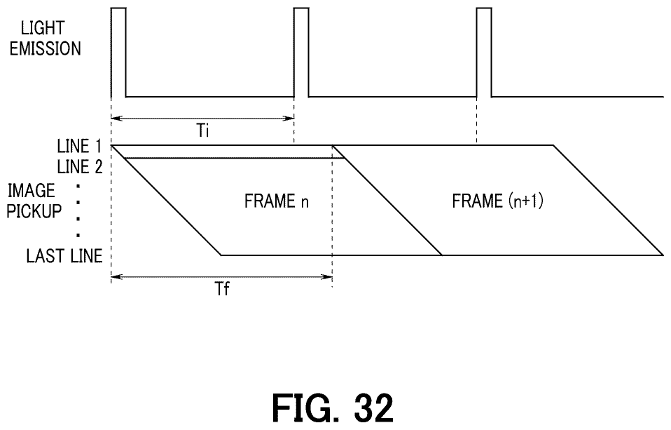

FIG. 32 is a timing chart for explaining the exposure unevenness between lines when the image pickup device 11 is a rolling shutter type in the additional technology 2.

When the image pickup device 11 is a rolling shutter type such as a CMOS imager, for example, the number of times of the pulse light emission in the exposure period is not necessarily the same depending on the line within one frame.

The FIG. 32 shows an example in which the light emission cycle Ti is shorter than the frame cycle Tf, that is, Ti<Tf. Focusing on the frame n, the pulse light emission is performed twice in the exposure period of the line 1, but the pulse light emission is performed only once in the exposure period of the last line, for example, resulting in the exposure unevenness between the lines.

The unevenness removal circuit 44 acquires the information of the pulse brightness L from the pulse brightness calculation circuit 43, and the exposure unevenness between frames as shown in FIG. 31 or between lines as shown in FIG. 32 is corrected by applying a gain based on a ratio of the brightnesses, for example.

According to the additional technology 2 shown in FIG. 28 to FIG. 32, by using the function formula that has a small error with the actual pulse brightness to calculate the pulse brightness L from the current control value I, the exposure unevenness between frames or between lines can be corrected with high accuracy.

Furthermore, by using the linear function formula, high-speed calculation can be performed with a small calculation load. Furthermore, by using a plurality of linear function formulas configured as the broken line, the correction accuracy of the exposure unevenness can be further improved.

### [Additional Technology 3]

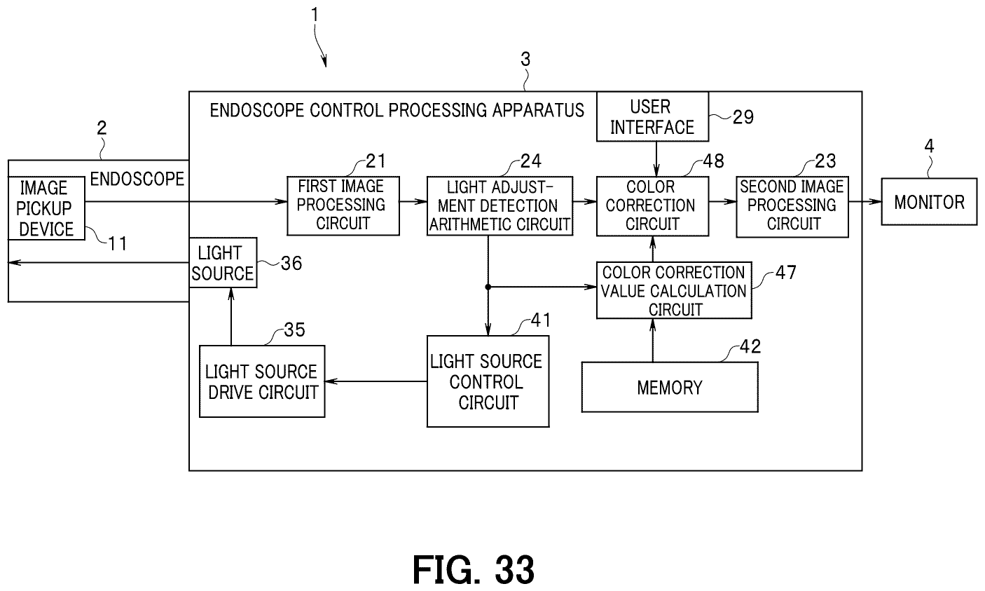

FIG. 33 is a view showing a configuration example of an endoscope control processing apparatus 3 serving also as a light source device in a additional technology 3. The additional technology 3 can have some relationship to the other technologies described herein, including that of the embodiments of FIGS. 1-18, and/or additional technology 1 and/or additional technology 2, and may include some or all of their constituent features, plus the additional features described.

For example, Japanese Patent Application Laid-Open Publication No. 2018-498 describes an endoscope system including a correction value setting section and a recording unit. The correction value setting section sets a white balance correction value. The recording unit records coefficients used in a function that indicates a relationship between the white balance correction value of reference light and the white balance correction value of other light. The endoscope system sets a new white balance correction value used in white balance correction of a reference light source. When switching from the reference light source to the other light source, the endoscope system calculates the white balance correction value of the other light source using the new white balance correction value and the coefficients. The endoscope system performs the white balance correction of the other light source using the calculated white balance correction value. Note that the publication describes xenon lamps, halogen lamps, metal halide lamps, LED lamps, and the like, as the light source.

Incidentally, even if the light source 36 is the same, the white balance (light emission color) may change depending on the light emission luminance of the light source 36.

Therefore, FIG. 33 shows a configuration example for correcting color changes when the light adjustment control value changes.

An endoscope system 1 includes an endoscope 2, the endoscope control processing apparatus 3, and a monitor 4 as described above.

The endoscope 2 is configured as an electronic endoscope as described above, and includes an image pickup device 11.

The endoscope control processing apparatus 3 includes the first image processing circuit 21, the second image processing circuit 23, the light adjustment detection arithmetic circuit 24, the user interface 29, the light source drive circuit 35, the light source 36, and the light source control circuit 41 that are described above. The endoscope control processing apparatus 3 further includes a memory 46, a color correction value calculation circuit 47, and a color correction circuit 48.

As described above, the light source 36 includes the light-emitting element 36a (see FIG. 20, etc.) configured as an LED, for example.

The memory 46 functions as a color correction value calculation parameter storage circuit, and stores color correction value calculation parameters in the non-volatile manner. Here, the color correction value calculation parameters are parameters for color correction of the image signal depending on the light adjustment control value. The memory 46 may be a part of the memory 30b shown in FIG. 3, or may be provided separately from the memory 30b.

The light adjustment detection arithmetic circuit 24 receives the image signal from the first image processing circuit 21, and creates a light adjustment detection value by adding and averaging luminance signals in a pixel area of the image pickup device 11 (image sensor) in which the optical image of the endoscope 2 is formed. Furthermore, the light adjustment detection arithmetic circuit 24 compares the light adjustment detection value with the light adjustment detection target value to create the light adjustment control value that determines the next light emission luminance of the light source 36 and output the light adjustment control value to the light source control circuit 41. The light source control circuit 41 linearly controls a light emission luminance of the light source 36 with respect to the light adjustment control value. At this time, the light adjustment control value generated by the light adjustment detection arithmetic circuit 24 is also transmitted to the color correction value calculation circuit 47. The color correction value calculation circuit 47 reads the color correction value calculation parameters from the memory 46, and calculates the color correction value from the light adjustment control value.

For example, it is assumed that the image signal is composed of a red (R) signal, a green (G) signal, and a blue (B) signal. Furthermore, it is assumed that the green (G) signal is multiplied by no gain for color correction. In this case, the color correction values correspond to a gain for color correction to the red (R) signal, and a gain for color correction to the blue (B) signal.

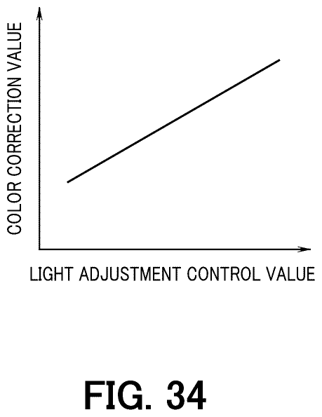

FIG. 34 is a graph showing an example of a function formula for calculating the color correction value from the light adjustment control value in the additional technology 3.

The color correction value calculation circuit 47 calculates the color correction value from the light adjustment control value as shown in FIG. 34. The function formula shown in FIG. 34 is a linear function formula, and is expressed by the following Equation 15 when the light adjustment control value is x, the color correction value is y, the inclination is u, and the intercept is v.

$$y = u \times x + v \qquad ( Equation 15 )$$

More specifically, when the inclination and the intercept for the red (R) signal are ur and vr, respectively, the color correction value for the red (R) signal yr (R gain) is obtained by the following Equation 16 using the light adjustment control value x.

$$yr = ur \times x + vr \qquad ( Equation 16 )$$

Similarly, when the inclination and the intercept for the blue (B) signal are ub and vb, respectively, the color correction value for the blue (B) signal yb (B gain) is obtained by the following Equation 17 using the light adjustment control value x.

$$yb = ub \times x + vb \qquad ( Equation 17 )$$

In the process inspection, spectral data when the light source 36 is caused to emit light at different light emission luminance is acquired, to calculate the inclination ur and the intercept yr for the red (R) signal and the inclination ub and the intercept vb for the blue (B) signal, using the acquired spectral data. As the spectral data, the B/G ratio, the R/G ratio, the peak wavelength, etc., may be acquired, for example.

Here, the light emission luminance of the light source 36 differs depending on the light emission mode of the illumination light. Specifically, in the strobe light emission mode, the light source 36 emits light for a short time with high light emission luminance due to a large current. In the WLI light emission mode, the light source 36 emits light with moderate light emission luminance (i.e., lower than that in the strobe light emission mode) due to medium current. In the NBI light emission mode, the light source 36 emits light with low light emission luminance (i.e., lower than that in the WLI light emission mode) due to small current.

Then, in the process inspection, the inclination ur and the intercept yr for the red (R) signal and the inclination ub and the intercept vb for the blue (B) signal are calculated as the color correction value calculation parameters, based on the spectral data acquired by causing the light source 36 to emit light in each light emission mode (in particular, the WLI light emission mode and the strobe light emission mode in which the subject is observed in the normal color).

Specifically, in the WLI light emission mode, the light adjustment control value x1, the R/G ratio RG1, and the B/G ratio BG1 are acquired. Furthermore, in the strobe light emission mode, the light adjustment control value x2, the R/G ratio RG2, and the B/G ratio BG2 are acquired.

Furthermore, the R/G ratio and the B/G ratio of the reference white light are denoted as RG0 and BG0, respectively. At this time, the following ratios r1, r2, b1, and b2 are respectively calculated.

$$r 1 = RG 1 / RG 0$$

$$r 2 = RG 2 / RG 0$$

$$b 1 = BG 1 / BG 0$$

$$b 2 = BG 2 / BG 0$$

Then, the inclination ur and the intercept yr in Equation 16 are calculated so as to pass through (x, yr)=(x1, r1) and (x, yr)=(x2, r2). Similarly, the inclination ub and the intercept vb in Equation 17 are calculated so as to pass through (x, yb)=(x1, b1) and (x, yb)=(x2, b2). The calculated color correction value calculation parameters are stored in the memory 46 in the non-volatile manner, as described above.

Note that the process inspection may be performed on each individual of the endoscope control processing apparatus 3. In addition, results of the process inspection performed on a representative individual of the endoscope control processing apparatus 3 may be applied to each individual of the same product.

The color correction circuit 48 is configured, for example, as a white balance circuit, a paint circuit, or the like. The color correction circuit 48 acquires from the color correction value calculation circuit 47 the color correction values yr and yb calculated based on the light adjustment control value x. The color correction circuit 48 performs the color correction by multiplying the red (R) signal by the color correction value yr (R gain), and the blue (B) signal by the color correction value yb (B gain).

Note that in the above description, the linear function formula is used, but it is not limited to this, and a quadratic or higher degree function formula may be used.

In addition, in the above description, the color correction value calculation circuit 47 reads and uses the color correction value calculation parameters stored in the memory 46, but it is not limited to this. For example, if the color correction value calculation circuit 47 preconfigured with an arithmetic circuit corresponding to the above-described function formula is used, the memory 46 may be omitted. For example, when the color correction value calculation parameters for each individual are used, the memory 46 may be used. In addition, when the color correction value calculation parameters common to each individual of the same product are used, either a configuration with or without the memory 46 may be adopted.

Furthermore, in the above description, the example has been given in which the color correction value is calculated from the light adjustment control value outputted from the light adjustment detection arithmetic circuit 24, but the color correction value may be calculated from the current control value I indicated by the current value control signal generated by the light source control circuit 41.

Note that the configuration of calculating the color correction value from the light adjustment control value, etc., as shown in Equation 15 to Equation 17 can be applied in all the light emission modes, but relatively high effects can be obtained even by adopting the following simple configuration.

That is, since the NBI light emission mode is not a mode for observing the subject with normal color balance, the color correction is omitted. In addition, in the WLI light emission mode, it is assumed that the color deviation of the image in the current drive range as shown in FIG. 21 is small. Therefore, the color correction is omitted even when the driving current changes in the WLI light emission mode. Furthermore, the light emitted in the WLI light emission mode is assumed to be the reference white light (that is, in the WLI light emission mode, the color correction values are assumed to be yr=yb=1), and the color correction is performed only in the strobe light emission mode. At this time, in the strobe light emission mode, the processing of calculating the color correction values yr and yb from the light adjustment control value, etc., as shown in Equation 16 and Equation 17, depending on the magnitude of the driving current (that is, depending on the light adjustment control value) is performed to perform the color correction by the color correction circuit 48.

According to the additional technology 3 shown in FIG. 33 to FIG. 34, for example, a loss of the white balance of the image caused by differences in spectral characteristics of the light source 36 in the WLI light emission mode and the strobe light emission mode can be corrected well.

Furthermore, in the same light emission mode (in particular, the strobe light emission mode), changes in the color of the image caused by changes in the spectral characteristics of the light source 36 due to the magnitude of the driving current depending on the light adjustment control can be corrected well.

### [Additional Technology 4]

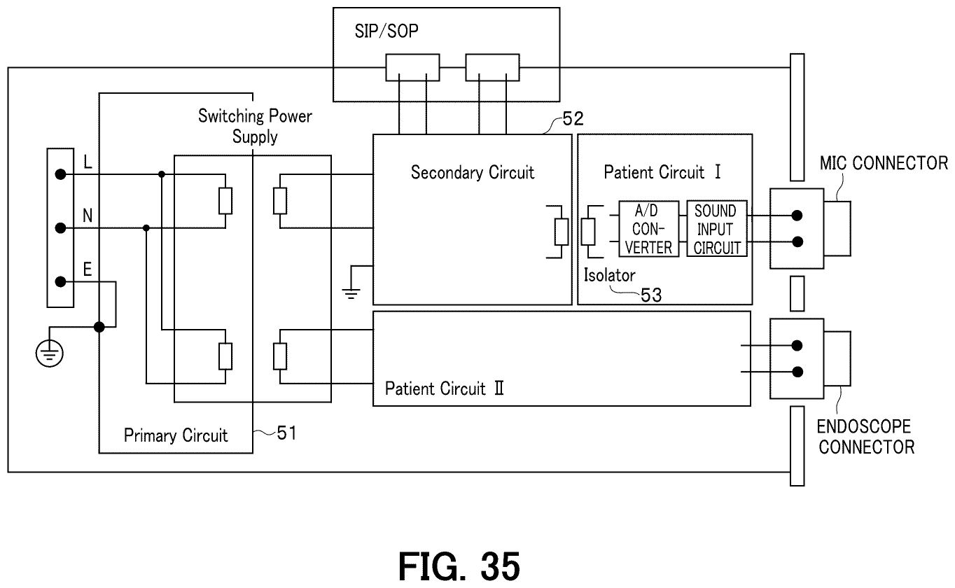

Furthermore, the present disclosure may be applied to an endoscope processor including the following configuration. FIG. 35 is a view showing a configuration example of the endoscope processor configured to supply power source for a sound input circuit using an isolator element 53 in a additional technology 4. The additional technology 4 can have some relationship to the other technologies described herein, including that of the embodiments of FIGS. 1-18, and/or additional technology 1, additional technology 2 and/or additional technology 3, and may include some or all of their constituent features, plus the additional features described.

The endoscope processor illustrated in FIG. 35 has, as a configuration for the endoscope, a configuration for supplying power source for the endoscope from a primary power source 51 (primary circuit), and, as a configuration for sound input, a configuration for supplying power source for a sound input circuit from a secondary power source 52 (secondary circuit) via the isolator element 53 with a built-in isolation transformer. In the configuration shown in FIG. 35, a sound signal is also transmitted to the secondary circuit using the isolator element 53.

The endoscope processor having the configuration as shown in FIG. 35 can be connected to an endoscope. Furthermore, the endoscope processor can be connected to a microphone for acquiring vocalized sounds from a patient. The endoscope processor can perform laryngo-stroboscopy. In the laryngo-stroboscopy, frequency components are extracted from the generated sounds acquired by using the microphone. Then, in the laryngo-stroboscopy, by performing lighting control of the strobe light in synchronization with the frequency of the vocal cord vibration, an observation with suppressed blurring of the vocal cord vibration. There are types of microphones that are placed in contact with the patient to acquire the generated sounds. Even when using such a type of the microphone, the configuration including the isolator element 53 as shown in FIG. 35 has effects that the patient is protected from an electric shock in an event of equipment failure, and the influence of noise on the sound signal can be reduced.

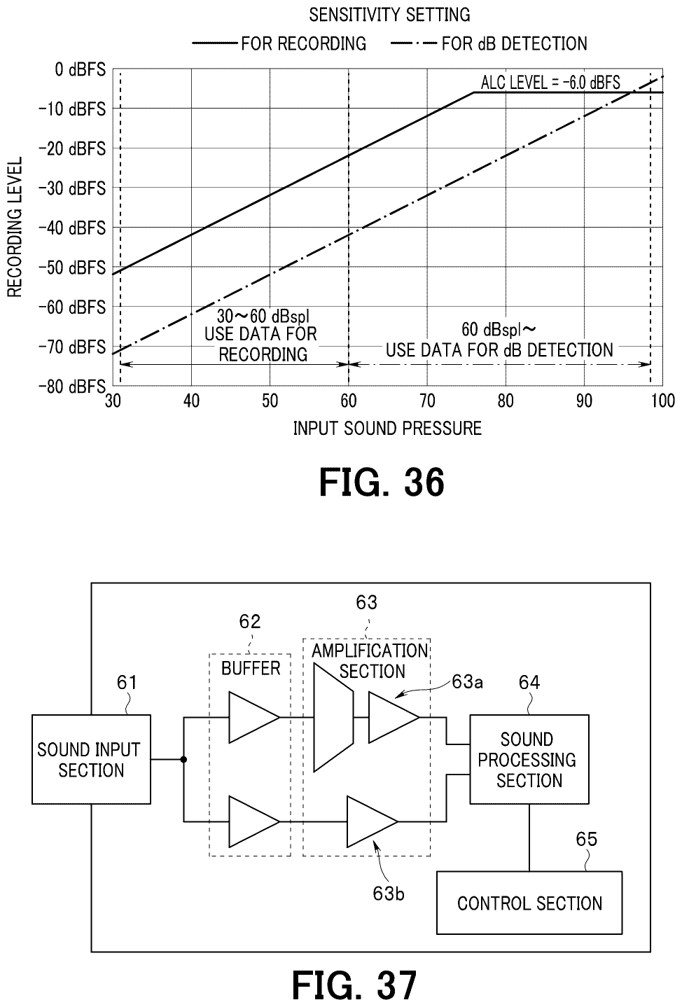

In addition, the endoscope processor capable of performing the laryngo-stroboscopy may include the following configuration. FIG. 36 is a graph for explaining that an amplification line with a large gain is used when an input sound pressure to the microphone is less than or equal to a threshold value, and an amplification line with a small gain is used when the input sound pressure is greater than the threshold value in the additional technology 4. FIG. 37 is a view showing a configuration example of the endoscope processor including a plurality of amplification mechanisms with different amplification degrees.

The endoscope processor illustrated in FIG. 37 includes a sound input section 61 that acquires the sound signal by receiving the generated sounds of the patient, the plurality of amplification mechanisms (amplification sections) 63 that are illustrated in FIG. 37 and amplify the acquired sound signal, a buffer circuit 62 that suppresses the influence of impedance differences due to the different amplification degrees, a sound processing section 64 that converts the sound signal from an analog signal to a digital signal, and a control section 65 that controls the sound processing section 64.

The amplification mechanisms 63 includes a first amplification mechanism 63a and a second amplification mechanism 63b. The first amplification mechanism 63a is for when the sound input signal is small, and has a gain larger than that of the second amplification mechanism 63b. The second amplification mechanism 63b is for when the sound input signal is large, and has a gain smaller than that of the first amplification mechanism 63a. The first amplification mechanism 63a has an automatic level control (ALC) function. The amplification mechanism having the ALC function reduces the amplification degree when an input higher than or equal to a specified level is received, thereby preventing clipping of the input sound. When measuring the loudness of the vocalized sound of the patient (dB detection), an amplification line is switched depending on the vocalized sound of the patient, i.e., the input sound pressure to the microphone, as shown in FIG. 36. Specifically, the amplification line passing through the first amplification mechanism 63a with the large gain is used when the input sound pressure is less than or equal to a threshold value, and the amplification line is switched to one passing through the second amplification mechanism 63b with the small gain when the input sound pressure becomes greater than the threshold. The amplification line passing through the first amplification mechanism 63a with the large gain has the ALC function as described above, which automatically reduces the gain when the input sound pressure is great. Therefore, it is not possible to use only the amplification line with the large gain for the dB detection.

The volume of the vocalized sound of the patient varies depending on the severity of the disease and the efficiency of the individual vocalization, resulting in a wide range of sound levels acquired by the endoscope apparatus. Furthermore, the volume of the sound signal acquired by the endoscope apparatus differs depending on the sensitivity of the microphone that acquires the sound. Therefore, if the collected sound is amplified with a single gain, the acquired sound may become too small or, conversely, may be amplified too much and clipped. However, by including the plurality of amplification mechanisms as described above, the sound input amount can be optimized.

In addition, the endoscope processor capable of performing the laryngo-stroboscopy may include the following configuration.

The endoscope processor includes the sound input section 61 that acquires the generated sound of the patient, a microphone detection section that detects connection of the microphone, and a circuit that blocks the sound signal output from the sound input section 61 when the microphone is not connected. The presence of the circuit that blocks the sound signal output from the sound input section 61 prevents noise from being recorded when the microphone is not connected and during recording of endoscope videos. In addition, the sound processing section 64 may be configured to determine the input sound level, and block the sound signal output when the input sound level is less than or equal to a certain value.

When the microphone is not connected, dark noise is amplified, resulting in loud noise being recorded. In particular, when the ALC function is installed, the gain is maximized when the microphone is not connected, causing the noise to become loud. However, by providing the circuit that blocks the sound signal output from the sound input section 61, the recording of noise can be prevented.

In addition, the endoscope processor capable of performing the laryngo-stroboscopy may include the following configuration.

The endoscope processor includes a sound input section 61 that acquires the generated sound of the patient, a setting section that selects connected device information, and a sound output section that outputs the signal-processed sound.

The line level of the sound (voltage level of the sound signal) is not standardized, but is designed at various voltage levels for each device. Therefore, the voltage level of the sound signal outputted by the endoscope apparatus is not always appropriate for a sound recording device. In contrast, by adopting the above-described configuration, the level of the output sound signal can be optimally adjusted depending on an external device (connected device) that is set (selected).

Note that the above description mainly describes a case where the present disclosure relates to the endoscope control processing apparatus and the endoscope system including the endoscope control processing apparatus, but the present disclosure is not limited thereto. For example, the present disclosure relates to the method of operating the endoscope control processing apparatus, or the endoscope apparatus. In addition, the present disclosure may relate to a computer program that causes a computer to perform processing similar to that of the endoscope control processing apparatus, and the like. Furthermore, the present disclosure may relate to a non-transitory computer-readable recording medium that records the computer program, or the like.

Here, some examples of the recording medium that stores a computer program product include a portable recording medium such as a flexible disk, a compact disc read only memory (CD-ROM), a digital versatile disc (DVD) or the like, or a recording medium such as a hard disk. It is not limited to the entirety of the computer program to be stored in the recording medium, but a part of the computer program may be stored in the recording medium. In addition, the entirety or a part of the computer program may also be distributed or provided via a communication network. When a user installs the computer program on the computer from the recording medium or downloads the computer program via the communication network and installs it on the computer, the computer program is read by the computer and all or a part of the operations are executed, to thereby enable the operations of the endoscope control processing apparatus, or the like as described above to be executed.

Furthermore, the present application is not limited as-is to the above-described embodiments. It is possible to embody the present embodiments by modifying the constituent elements in a range without departing from the gist of the present application at the practical stage. In addition, various aspects of the present application can be achieved by appropriately combining the plurality of constituent elements disclosed in the above-described embodiments. Some of the constituent elements may be deleted from all the constituent elements disclosed in the embodiment, for example. Furthermore, constituent elements over different embodiments may be combined as appropriate. It goes without saying that various modifications and applications can be implemented within a range without departing from the gist of the present application.

What is claimed is:

1. An endoscope control processing apparatus that (a) acquires image signals in frames from an endoscope including an image pickup device having an electronic shutter function configured to control an exposure time, and (b) processes the image signals, the endoscope control processing apparatus comprising a processor,
the processor configured to:
perform light adjustment detection on an image signal acquired in a first frame to calculate an exposure time target value for a second frame that is later in time than the first frame;
calculate an electronic shutter control value for the second frame based on the exposure time target value;
calculate an actual exposure time from the electronic shutter control value;
calculate an exposure time ratio between the actual exposure time and the exposure time target value;
cause the image pickup device to pick up an image based on the electronic shutter control value, in order to acquire an image signal in the second frame from the endoscope; and
adjust a gain based on the exposure time ratio in image processing on the image signal in the second frame.

2. The endoscope control processing apparatus according to claim 1, wherein
the processor is configured to:
compare the electronic shutter control value with a threshold value; and
determine whether or not to adjust the gain based on the exposure time ratio, depending on whether the electronic shutter control value is greater or less than the threshold value.

3. The endoscope control processing apparatus according to claim 2, wherein:
the image pickup device is configured to control an electronic shutter in line units;
the electronic shutter control value is a number of lines that are light-shielded during exposure; and
the processor is configured:
to adjust the gain based on the exposure time ratio in the image processing on the image signal in the second frame when the electronic shutter control value is determined to be greater than or equal to the threshold value; and
not to adjust the gain based on the exposure time ratio in the image processing on the image signal in the second frame when the electronic shutter control value is determined to be less than the threshold value.

4. The endoscope control processing apparatus according to claim 3, wherein the threshold value is set as a value less than or equal to a value obtained by subtracting the quotient of 100 divided by a target light adjustment accuracy from a total number of lines of the image pickup device.

5. An endoscope system comprising:
an endoscope including an image pickup device having an electronic shutter function configured to control an exposure time; and
an endoscope control processing apparatus including a processor configured to acquire image signals in frames from the endoscope and to process the image signals, wherein
the processor is configured to:
perform light adjustment detection on an image signal acquired in a first frame to calculate an exposure time target value for a second frame that is later in time than the first frame;
calculate an electronic shutter control value for the second frame based on the exposure time target value;
calculate an actual exposure time from the electronic shutter control value;
calculate an exposure time ratio between the actual exposure time and the exposure time target value;
cause the image pickup device to pick up an image based on the electronic shutter control value, in order to acquire an image signal in the second frame from the endoscope; and
adjust a gain based on the exposure time ratio in image processing on the image signal in the second frame.

6. The endoscope system according to claim 5, wherein
the processor is configured to:
compare the electronic shutter control value with a threshold value; and
determine whether or not to adjust the gain based on the exposure time ratio, depending on whether the electronic shutter control value is greater or less than the threshold value.

7. The endoscope system according to claim 6, wherein:
the image pickup device is configured to control an electronic shutter in line units;
the electronic shutter control value is a number of lines that are light-shielded during exposure; and
the processor is configured:
to adjust the gain based on the exposure time ratio in the image processing on the image signal in the second frame when the electronic shutter control value is determined to be greater than or equal to the threshold value; and
not to adjust the gain based on the exposure time ratio in the image processing on the image signal in the second frame when the electronic shutter control value is determined to be less than the threshold value.

8. The endoscope system according to claim 7, wherein the threshold value is set as a value less than or equal to a value obtained by subtracting the quotient of 100 divided by a target light adjustment accuracy from a total number of lines of the image pickup device.

9. A method of operating an endoscope control processing apparatus that (a) acquires image signals in frames from an endoscope including an image pickup device having an electronic shutter function configured to control an exposure time, and (b) processes the image signals, the method comprising:
performing light adjustment detection on an image signal acquired in a first frame to calculate an exposure time target value for a second frame that is later in time than the first frame;
calculating an electronic shutter control value for the second frame based on the exposure time target value;
calculating an actual exposure time from the electronic shutter control value;
calculating an exposure time ratio between the actual exposure time and the exposure time target value;
causing the image pickup device to pick up an image based on the electronic shutter control value, in order to acquire an image signal in the second frame from the endoscope; and
adjusting a gain based on the exposure time ratio in image processing on the image signal in the second frame.

10. The method of operating the endoscope control processing apparatus according to claim 9, further comprising:
comparing the electronic shutter control value with a threshold value; and
determining whether or not to adjust the gain based on the exposure time ratio, depending on whether the electronic shutter control value is greater or less than the threshold value.

11. The method of operating the endoscope control processing apparatus according to claim 10, wherein:
the image pickup device is capable of controlling an electronic shutter in line units;
the electronic shutter control value is a number of lines that are light-shielded during exposure; and
the determining whether or not to adjust the gain based on the exposure time ratio, depending on whether the electronic shutter control value is greater or less than the threshold value includes:
adjusting the gain based on the exposure time ratio in the image processing on the image signal in the second frame when the electronic shutter control value is greater than or equal to the threshold value; and
not adjusting the gain based on the exposure time ratio in the image processing on the image signal in the second frame when the electronic shutter control value is less than the threshold value.

12. The method of operating the endoscope control processing apparatus according to claim 11, wherein the threshold value is set as a value less than or equal to a value obtained by subtracting the quotient of 100 divided by a target light adjustment accuracy from a total number of lines of the image pickup device.

13. A non-transitory computer-readable recording medium that records a program configured to cause a computer that (a) acquires image signals in frames from an endoscope including an image pickup device having an electronic shutter function configured to control an exposure time and (b) processes the image signals, to perform endoscope control processing,
the endoscope control processing comprising:
performing light adjustment detection on an image signal acquired in a first frame to calculate an exposure time target value for a second frame that is later in time than the first frame;
calculating an electronic shutter control value for the second frame based on the exposure time target value;
calculating an actual exposure time from the electronic shutter control value;
calculating an exposure time ratio between the actual exposure time and the exposure time target value;
causing the image pickup device to pick up an image based on the electronic shutter control value, in order to acquire an image signal in the second frame from the endoscope; and
adjusting a gain based on the exposure time ratio in image processing on the image signal in the second frame.

14. The non-transitory computer-readable recording medium according to claim 13, wherein the endoscope control processing further comprises:
comparing the electronic shutter control value with a threshold value; and
differentiating whether or not to adjust the gain based on the exposure time ratio, depending on whether the electronic shutter control value is greater or less than the threshold value.

15. A hardware processor of an endoscope apparatus,
the processor configured to:
generate a first image signal by performing image processing on a first image pickup signal, the first image pickup signal being generated by an image pickup device reading a charge corresponding to a first frame;
perform light adjustment detection on the first image signal to determine a target exposure time in a second frame that is a frame later than the first frame;
determine an electronic shutter control value for adjusting an exposure time in the second frame based on the target exposure time;
calculate an exposure time ratio that is a ratio between an actual exposure time adjusted by the electronic shutter control value and the target exposure time in the second frame;
generate a second image signal by performing image processing on a second image pickup signal, the second image pickup signal being generated by the image pickup device reading a charge corresponding to the second frame, the image pickup device having been controlled based on the electronic shutter control value in the second frame; and
adjust a gain based on the calculated exposure time ratio in the second frame in the image processing on the second image pickup signal.

16. The endoscope apparatus according to claim 15, wherein:
the processor is configured:
to compare the electronic shutter control value with a threshold value;
to apply gain adjustment based on the exposure time ratio when the comparison indicates that the electronic shutter control value is greater than the threshold value; and
not to apply the gain adjustment based on the exposure time ratio when the comparison indicates that the electronic shutter control value is less than the threshold value.
